SEQUENCE LISTING (19 sequences)

SEQ ID NO: 1 - protein, 167 aa, Mus musculus
MCWRPLCRFLWLWSYLSYVQAVPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 2 - protein, 7 aa, Mus musculus
SCSLPQT

SEQ ID NO: 3 - protein, 15 aa, Mus musculus
AVPIQKVQDDTKTLI

SEQ ID NO: 4 - protein, 15 aa, Mus musculus
TKTLIKTIVTRINDI

SEQ ID NO: 5 - protein, 15 aa, Mus musculus
RINDISHTQSVSAKQ

SEQ ID NO: 6 - protein, 15 aa, Mus musculus
VSAKQRVTGLDFIPG

SEQ ID NO: 7 - protein, 15 aa, Mus musculus
DFIPGLHPILSLSKM

SEQ ID NO: 8 - protein, 15 aa, Mus musculus
SLSKMDQTLAVYQQV

SEQ ID NO: 9 - protein, 15 aa, Mus musculus
VYQQVLTSLPSQNVL

SEQ ID NO: 10 - protein, 15 aa, Mus musculus
SQNVLQIANDLENLR

SEQ ID NO: 11 - protein, 15 aa, Mus musculus
DLLHLLAFSKSCSLP

SEQ ID NO: 12 - protein, 15 aa, Mus musculus
SCSLPQTSGLQKPES

SEQ ID NO: 13 - protein, 15 aa, Mus musculus
QKPESLDGVLEASLY

SEQ ID NO: 14 - protein, 15 aa, Mus musculus
EASLYSTEVVALSRL

SEQ ID NO: 15 - protein, 15 aa, Mus musculus
ALSRLQGSLQDILQQ

SEQ ID NO: 16 - protein, 12 aa, Mus musculus
DILQQLDVSPEC

SEQ ID NO: 17 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 18 - protein, 7 aa, Homo sapiens
SCHLPWA

SEQ ID NO: 19 - protein, 18 aa, Homo sapiens
VTGLDFIPGLHPILTLSK

(19) United States
(12) Patent Application Publication
(10) Pub. No.: US 2011/0136728 A1
(43) Pub. Date: Jun. 9, 2011

(54) METHODS OF INCREASING BONE FORMATION USING LEPTIN-RELATED PEPTIDES

(76) Inventors: Patricia Grasso, Newtown, CT (US); Matthew C. Leinung, Albany, NY (US)

(21) Appl. No.: 12/964,389

(22) Filed: Dec. 9, 2010

Related U.S. Application Data
(60) Provisional application No. 61/285,105, filed on Dec. 9, 2009.
(60) Provisional application No. 61/296,768, filed on Jan. 20, 2010.
(60) Provisional application No. 61/303,088, filed on Feb. 10, 2010.

(51) Int. Cl. A61K 38/08; A61P 3/10; A61P 35/00; A61P 19/00; A61P 31/06; A61P 31/18; A61P 1/12

(52) U.S. Cl. 514/2.4; 514/16.7; 514/21.7; 514/19.3; 514/6.9; 514/16.9; 514/4.1

(57) ABSTRACT

The present invention relates to methods of increasing bone formation in patient suffering from a wasting disorder by orally or intranasally administering a pharmaceutically effective amount of a leptin peptide and a pharmaceutically acceptable carrier, wherein the leptin peptide increases serum osteocalcin levels.

### RELATED APPLICATIONS

This application claims the benefit of priority to U.S. Provisional Application No. 61/285,105, filed Dec. 9, 2009, U.S. Provisional Application No. 61/296,768, filed Jan. 20, 2010, and U.S. Provisional Application No. 61/303,088, filed Feb. 10, 2010, the entire contents of which are incorporated herein by reference in their entireties.

### REFERENCE TO A “SEQUENCE LISTING”

The sequence listing material in the text file entitled “29708_{—}504001US_Sequence_Listing_ST25.txt” (7,090 bytes), which was created on Dec. 8, 2010, is herein incorporated by reference in its entirety.

### FIELD OF THE INVENTION

The present invention relates to methods of increasing bone formation in a subject by administering a leptin peptide, wherein the leptin peptide increases serum osteocalcin levels.

### BACKGROUND OF THE INVENTION

Results of earlier preclinical studies with mouse [D-Leu-4]-OB3, a synthetic peptide amide with leptin-like activity, have shown that intraperitoneal (ip) delivery of this peptide significantly improves a number of metabolic dysfunctions including, for example, obesity and elevated blood glucose, which is associated with the obesity syndrome in the ob/ob mouse model. (See Rozhayskaya-Arena M. et al., Endocrinology 141:2501-2517 (2000) and Grasso P. et al., Regulatory Pep. 101:123-129 (2001)). However, ip administration of leptin-related peptides is often accompanied by un-desirable side effects such as discomfort and risk of infection. Thus, there is a need to administer leptin-related peptides using methods other than ip delivery.

### SUMMARY OF THE INVENTION

Intranasal or oral delivery of mouse [D-Leu-4]-OB3 with a transmucosal absorption enhancing agent, results in significantly higher bioavailability of mouse [D-Leu-4]-OB3 when compared to ip and other commonly used injection methods of drug delivery. For example, oral or intranasal delivery of [D-Leu-4]-OB3 in Intravail® provides a convenient, non-threatening, and non-invasive approach to the clinical management of human obesity, type 2 diabetes, and osteoporosis resulting from anorexia nervosa, and other wasting diseases. This approach eliminates the discomfort and risk of infection which can accompany needle-stick injuries, as well as the expense and inconvenience associated with the appropriate collection, transport, and disposal of used needles and syringes. [D-Leu-4]-OB3 (SEQ ID NO:24) is a small peptide, seven amino acids in length that is easily synthesized and relatively inexpensive because of its small size.

Disclosed herein are methods of decreasing bone loss (and/or increasing bone turnover) by administering to a subject in need thereof a therapeutically effective amount of a pharmaceutical composition containing a leptin peptide of SEQ ID NO:2 or SEQ ID NO:18 and a pharmaceutically acceptable carrier, wherein the leptin peptide increases serum osteocalcin levels, and wherein the increase in serum osteocalcin levels is a specific and sensitive marker for increased bone formation. In various embodiments, the step of administering to a subject can be through oral, anal, injection, and/or intranasal administration. Preferably, the subject is a mammal, e.g. a primate, rodent, feline, canine, domestic livestock (such as cattle, sheep, goats, horses and pigs). Most preferably, the mammal is a human.

The pharmaceutically acceptable carrier can be a drug delivery system, for example, a transmucosal absorption enhancer. For example, the transmucosal absorption enhancer is Intravail®.

In various embodiments, the leptin peptide is a purified peptide which is an OB-3 peptide of amino acid residues ¹¹⁶Ser-Cys-Ser-Leu-Pro-Gln-Thr¹²² of mouse leptin protein (SEQ ID NO:2) or ¹¹⁶Ser-Cys-His-Leu-Pro-Trp-Ala¹²² of human leptin protein (SEQ ID NO:18). Further, one, two, three, four, five, six or seven amino acids of the leptin peptide used in the pharmaceutical composition can be substituted with any of its corresponding D-amino acid isoform.

Any of the methods disclosed herein are used to treat subjects suffering from a disorder selected from the group consisting of malnutrition, starvation, anorexia nervosa, osteoporosis, cancer, diabetes, tuberculosis, chronic diarrhea, AIDS, and Superior mesenteric artery syndrome.

Also disclosed herein are methods of treating a wasting disease in a subject by administering to the subject suffering therefrom a therapeutically effective amount of a pharmaceutical composition containing a leptin peptide of SEQ ID NO:2 or SEQ ID NO:18 and a pharmaceutically acceptable carrier, wherein the leptin peptide increases serum osteocalcin levels in said subject. By way of non-limiting example, the wasting disease is selected from the group consisting of malnutrition, starvation, anorexia nervosa, osteoporosis, cancer, diabetes, tuberculosis, chronic diarrhea, AIDS, and/or Superior mesenteric artery syndrome. Those skilled in the art will recognize that the step of administering to a subject suffering from the wasting disease can be anal, oral or intranasal administration (or a combination thereof). Moreover, the pharmaceutical composition is in the form of a capsule, a tablet, a quick dissolving film, a liquid, nose-drops, a spray, and/or a suppository.

In certain embodiments, the leptin peptide used in these methods are purified peptides which is an OB-3 peptide amino acid residues ¹¹⁶ Ser-Cys-Ser-Leu-Pro-Gln-Thr¹²² of mouse leptin protein (SEQ ID NO:2) or ¹¹⁶Ser-Cys-His-Leu-Pro-Trp-Ala¹²² of human leptin protein (SEQ ID NO:18). Further, any one, two, three, four, five, six or seven amino acids of these leptin peptide can substituted with its corresponding D-amino acid isoform.

Preferably, the pharmaceutically acceptable carrier used in these methods is a drug delivery system, for example a transmucosal absorption enhancer. Particularly, the transmucosal absorption enhancer is Intravail®.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims. The citation of any reference herein should not be deemed as an admission that such reference is available as prior art to the instant invention.

### DESCRIPTION OF THE FIGURES

FIG. 1 is a representation of the primary structure of mouse leptin protein (SEQ ID NO:1), wherein the letters indicate the one-letter designation for amino acid residues, and the lines encompass the amino acid residues of various leptin-related peptides.

FIG. 2 is a representation of the primary structure of human leptin (SEQ ID NO:17), wherein the letters indicate the one-letter designation for amino acid residues, and residues 116-122 are underlined.

FIGS. 3A and 3B are graphs that show the effects of mouse [D-Leu-4]-OB3 (1 mg/day, 10 days, gavage) on body weight gain in male C57BL/6J wild type (A) and ob/ob (B) mice allowed food and water ad libitum. The graph shows the changes in body weight (expressed as percent of initial weight) in mice treated with Intravail® alone or with mouse [D-Leu-4]-OB3 reconstituted in Intravail®. Each point represents the mean±SEM change in body weight for a group of six mice.

FIG. 4 is a graph that shows the effects of mouse [D-Leu-4]-OB3 (1 mg/day, 10 days, gavage) on daily food intake by male C57BL/6J wild type and ob/ob mice allowed food and water ad libitum. The graph shows the effects of Intravail® alone or of mouse [D-Leu-4]-OB3 reconstituted in Intravail® on daily food intake. Each bar represents food consumed per mouse per day (mean±SEM; n=6).

FIG. 5 is a graph that shows the effects of mouse [D-Leu-4]-OB3 (1 mg/day, 10 days, gavage) on daily water intake by male C57BL/6J wild type and ob/ob mice allowed food and water ad libitum. The graph shows the effects of Intravail® alone or of mouse [D-Leu-4]-OB3 reconstituted in Intravail® on daily water consumption. Each bar represents water consumed per mouse per day (mean±SEM; n=6).

FIGS. 6A and 6B are graphs that show the effects of mouse [D-Leu-4]-OB3 (1 mg/day, 10 days, gavage) on serum glucose levels in male C57BL/6J wild type (A) and ob/ob (B) mice allowed food and water ad libitum. The graph shows serum glucose levels at the beginning of the study (day 0) and after 10 days of treatment (day 11) with Intravail® alone or with mouse [D-Leu-4]-OB3 reconstituted in Intravail®. Each bar and vertical line represents mean±SEM serum glucose level (n=6).

FIG. 7 is a graph that shows the effects of mouse [D-Leu-4]-OB3 (1 mg/day, 10 days, gavage) on serum osteocalcin levels in male C47BL/6J wild type and ob/ob mice allowed food and water ad libitum. The graph shows the effect of Intravail® alone or of mouse [D-Leu-4]-OB3 reconstituted in Intravail® on serum osteocalcin. Each bar and vertical line represents mean±SEM serum osteocalcin level (n=6).

FIGS. 8A and 8B are graphs that show the effects of mouse [D-Leu-4]-OB3 (1 mg/day, 10 days, gavage) on body weight gain in calorie restricted (40%) male C57BL/6J wild type (A) and ob/ob (B) mice. The graph shows the changes in body weight (expressed as percent of initial weight) in mice treated with Intravail® alone or with mouse [D-Leu-4]-OB3 reconstituted in Intravail®. Each point represents the mean±SEM change in body weight for a group of six mice.

FIGS. 9A and 9B are graphs that show the effects of mouse [D-Leu-4]-OB3 (1 mg/day, 10 days, gavage) on serum glucose levels in calorie restricted (40%) male C57BL/6J wild type (A) and ob/ob (B) mice allowed food and water ad libitum. The graph shows serum glucose levels at the beginning of the study (day 0) and after 10 days of treatment (day 11) with Intravail® alone or with mouse [D-Leu-4]-OB3 reconstituted in Intravail®. Each bar and vertical line represents mean±SEM serum glucose level (n=6).

FIG. 10 is a graph that shows the effects of mouse [D-Leu-4]-OB3 (1 mg/day, 10 days, gavage) on serum osteocalcin levels in calorie restricted male C57BL/6J wild type and ob/ob mice. The graph shows the effect of Intravail® alone or of mouse [D-Leu-4]-OB3 reconstituted in Intravail® on serum osteocalcin. Each bar and vertical line represents mean±SEM serum osteocalcin level (n=6).

FIG. 11 is a graph that shows serum concentrations of mouse [D-Leu-4]-OB3 10, 30, 50, 70, 90 and 120 min after oral delivery of 1 mg of peptide by gavage to male Swiss Webster mice (n=6 mice per time point). Each value represents mean±SEM. Error bars are contained within the point, and ranged between 0.01 and 0.10 ng/ml.

FIG. 12 is a graph that shows the effects of mouse [D-Leu-4]-OB3 in 0.18% Intravail A5 (1 mg/day, 14 days) on body weight gain in male C57BLK/6-m db/db mice following intranasal administration.

FIG. 13 is a graph that shows the effects of mouse [D-Leu-4]-OB3 in 0.18% Intravail A5 (1 mg/day, 14 days) on food and water intake in male C57BLK/6-m db/db mice following intranasal administration.

FIG. 14 is a graph that shows the effects of mouse [D-Leu-4]-OB3 in 0.18% Intravail A5 (1 mg/day, 14 days) on serum osteocalcin in male C57BLK/6-m db/db mice following intranasal administration.

FIG. 15 is a graph that shows the effects of mouse [D-Leu-4]-OB3 in 0.18% Intravail A5 (1 mg/day, 14 days) on serum insulin in male C57BLK/6-m db/db mice following intranasal administration.

FIG. 16 is a graph that shows the effects of intranasal administration of mouse [D-Leu-4]-OB3 (1 mg/day, 14 days) on serum glucose levels in C57BLK/6-m db/db mice. The graph shows serum glucose levels at the beginning of the study (day 0) and after 14 days of treatment (day 14) with Intravail® or mouse [D-Leu-4]-OB3 reconstituted in Intravail®. Each bar and vertical line represents mean±SEM serum glucose level (n=6).

FIG. 17. Effects of intranasal administration of mouse [D-Leu-4]-OB3 (1 mg/day, 14 days) on body weight gain in C57BLk/6-m db/db mice. The graph shows the changes in body weight (expressed as percent of initial weight) in mice treated with Intravail® or mouse [D-Leu-4]-OB3 reconstituted in Intravail®. Each point represents the mean±standard error of mean change in body weight for a group of six mice.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. Unless expressly stated otherwise, the techniques employed or contemplated herein are standard methodologies well known to one of ordinary skill in the art. The examples of embodiments are for illustration purposes only. All patents and publications cited in this specification are incorporated herein by reference.

Therefore, if appearing herein, the following terms shall have the definitions set forth below. As used herein, “physiological obesity” and “physiologically obese” refer to excessive adipose tissue that is due at least in part to abnormalities in the endogenous leptin pathway, including abnormalities in the effective signaling initiated by the binding of leptin to the leptin receptor. Abnormalities in the endogenous leptin pathway may be manifested in a number of ways including an abnormal food intake, an abnormal activity level, or an abnormal body temperature. In addition, the present invention allows drugs to be identified which can modulate body mass completely independently of any inherent abnormality in the endogenous leptin pathway per se by augmenting or diminishing the natural effect of leptin.

As used herein, “leptin” encompasses biologically active variants of naturally occurring leptin, as well as biologically active fragments of naturally occurring leptin and variants thereof, and combinations of the preceding. Leptin is the polypeptide product of the ob gene as described in the International Patent Publication No. WO 96/05309, and the U.S. Pat. No. 6,309,853, each of which is incorporated herein by reference in its entirety. Putative analogs and fragments of leptin are reported in U.S. Pat. No. 5,521,283 and U.S. Pat. No. 5,532,336; and International Patent Publication No. PCT/US96/22308 and PCT Publication No. WO/1996/022308, each of which is incorporated herein by reference in its entirety.

As used herein the terms “bound” or “binds” or “associates” or “associated” are meant to include all such specific interactions that result in two or more molecules showing a preference for one another relative to some third molecule. This includes processes such as covalent, ionic, hydrophobic and hydrogen bonding but does not include non-specific associations such as solvent preferences.

As used herein, the phrase “conditions related to abnormalities of the endogenous leptin pathway” encompasses conditions and diseases due, at least in part, to abnormalities involving leptin as detailed above.

A “patient,” “individual,” “subject” or “host” refers to either a human or a non-human animal.

The term “mammal” is known in the art and includes humans, primates, bovines, porcines, canines, felines, and rodents (e.g., mice and/or rats).

As used herein, the term “pharmaceutically-acceptable salt” is art-recognized and refers to the relatively non-toxic, inorganic and organic acid addition salts of compounds, including, for example, those contained in the compositions described herein.

The term “pharmaceutically acceptable carrier” is art-recognized and refers to a pharmaceutically-acceptable material, composition or vehicle, such as, for example, a liquid or solid filler, diluent, excipient, solvent or encapsulating material, involved in carrying or transporting any subject composition or component thereof from one organ or portion of the body, to another organ or portion of the body. Each carrier must be “acceptable” in the sense of being compatible with the subject composition and its components and not injurious to the patient. Some non-limiting examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally”, as used herein, are all art-recognized and refer to the administration of a subject composition, therapeutic or other material other than directly into the central nervous system, such that it enters the patient's system and, thus, is subject to metabolism and other like processes.

Likewise, the terms “parenteral administration” and “administered parenterally” are also art-recognized and refer to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and/or intrasternal injection and infusion.

As used herein, “treating” a condition or disease refers to curing as well as ameliorating at least one symptom of the condition or disease.

The term “therapeutic agent” is art-recognized and refers to any chemical moiety that is a biologically, physiologically, or pharmacologically active substance that acts locally or systemically in a subject. This term also refers to any substance intended for use in the diagnosis, cure, mitigation, treatment or prevention of disease or in the enhancement of desirable physical or mental development and/or conditions in an animal or human.

Moreover, the term “therapeutic effect” is art-recognized and refers to a local or systemic effect in animals, particularly mammals, and more particularly humans, caused by a pharmacologically active substance. The phrase “therapeutically-effective amount” means that amount of such a substance that produces some desired local or systemic effect at a reasonable benefit/risk ratio and is applicable to any treatment. The therapeutically effective amount of such substance will vary depending upon the subject and disease or condition being treated, the weight and age of the subject, the severity of the disease or condition, the manner of administration and the like, which can readily be determined by one of ordinary skill in the art. For example, certain compositions described herein may be administered in a sufficient amount to produce a desired effect at a reasonable benefit/risk ratio applicable to such treatment.

The term “synthetic” is art-recognized and refers to production by in vitro chemical or enzymatic synthesis.

The term “medically-assisting” is used herein as a manner of attending to the health care needs of a subject who has a particular problem (e.g., an abnormality in the endogenous leptin pathway) which encompasses either diagnosing or treating that problem, and all combinations thereof. In one embodiment, the invention provides for medically assisting a mammalian subject suffering from an abnormality in the endogenous leptin pathway resulting in decreased leptin level or activity. In another embodiment, a mammalian subject may be suffering from an abnormality resulting in increased leptin level or activity. In each case, the decreased or increased leptin activity may be manifested as a pathological state.

“Variant” refers to a polynucleotide or polypeptide differing from the polynucleotide or polypeptide of the present invention, but retaining essential properties thereof. Generally, variants are overall closely similar, and in many regions, identical to the polynucleotide or polypeptide of the present invention. The variants may contain alterations in the coding regions, non-coding regions, or both.

As utilized herein, the term “functionally active” refers to species displaying one or more known functional attributes of a full-length leptin.

As utilized herein, the term “pharmaceutically acceptable” means a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans.

The term “carrier” refers to a diluent, adjuvant, excipient, or vehicle with which the therapeutic is administered and includes, but is not limited to such sterile liquids as water and oils. The characteristics of the carrier will depend on the route of administration.

As used herein, the term “therapeutically effective amount” means the total amount of each active component of the pharmaceutical composition or method that is sufficient to show a meaningful patient benefit, i.e., treatment, healing, prevention, and/or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

As used herein, “wasting” refers to the process by which a debilitating disease causes muscle and fat tissue to “waste” away. Wasting can be caused by an extremely low energy intake (e.g., caused by famine), nutrient losses due to infection, or a combination of low intake and high loss. Also used herein, “wasting diseases” are infections, disease, disorders, or conditions associated with wasting, and include, but are not limited to, tuberculosis, chronic diarrhea, AIDS, osteoporosis, cancer and/or Superior mesenteric artery syndrome. The mechanism may involve cachectin, also called tumor necrosis factor, a macrophage-secreted cytokine. Voluntary weight loss and eating disorders, such as anorexia nervosa, are also considered to be a wasting disease, as defined herein.

Intravail® (Aegis Therapeutics, San Diego, Calif.) is a patented transmucosal absorption enhancer that comprises a broad class of chemically synthesizable transmucosal absorption enhancement agents that allow non-invasive systemic delivery of potent peptide, protein, nucleotide-related, and other small and large molecule drugs that were previously only deliverable by injection. (See U.S. Pat. No. 5,661,130; U.S. Pat. No. 7,425,542; European Patent No. EP1789075; PCT Publication No. WO95/00151; U.S. Publication No. 2006/0046969; U.S. Publication No. 2006/0046962; U.S. Publication No. 2006/0045869; U.S. Publication No. 2006/0045868; U.S. Publication No. 2008/0268032; U.S. Publication No. 2008/0194461; U.S. Publication No. 2008/0200418; U.S. Publication No. 2008/0299079; PCT Publication No. WO 2009/029543; and U.S. Publication No. 2009/0047347, each of which are incorporated herein by reference in their entireties).

Intravail® absorption enhancement agents are mild and non-irritating to mucosal membranes. Moreover, these agents are safe, odorless, tasteless, non-toxic, non-irritating, non-denaturing, and non-mutagenic, chemically synthesized molecules that metabolize to CO₂ and H₂O. In fact, these molecules are closely related to mild surfactants, which are widely used in personal care and food products in significantly higher concentrations than those used in Intravail® formulations and are recognized as GRAS (Generally Regarded As Safe) substances for many applications. The use of Intravail® absorption enhancement agents exhibits a high degree of bioavailability, which is comparable to subcutaneous injection, via intranasal, buccal, intestinal, and other mucosal membrane administration routes. Thus, these agents can be used to deliver potent peptide, protein, and large molecule drugs that typically have only been delivered intraperitoneally (e.g. by injection).

In some embodiments, the compositions of the present embodiments comprise at least one low molecular weight leptin related peptide of the present embodiments and at least one alkylglycoside.

In some embodiments, pharmaceutical compositions are provided comprising at least one low molecular weight leptin related peptide of the present embodiments and a suitable nontoxic, nonionic alkylglycoside having a hydrophobic alkyl joined by a linkage to a hydrophilic saccharide. In some embodiments, the alkyl has from 9 to 24 carbons. In some embodiments, the alkyl has from 9 to 14 carbon atoms. In some embodiments, the saccharide is selected from the group consisting of maltose, sucrose and glucose. In some embodiments, the alkylglycoside further has a hydrophile-lipophile balance number in the range of about 10 to 20. In some embodiments, the linkage is selected from the group consisting of a glycosidic linkage, a thioglycosidic linkage, an amide linkage, a ureide linkage and an ester linkage. In some embodiments, the alkylglycoside has a concentration in the range of about 0.01% to 1.0%.

In some embodiments, pharmaceutical compositions are provided comprising at least one low molecular weight leptin related peptide of the present embodiments; a buffering agent; and an alkylglycoside; wherein the alkylglycoside is selected from the group consisting of dodecyl maltoside, tridecyl maltoside, sucrose mono-dodecanoate, sucrose mono-tridecanoate, and sucrose mono-tetradecanoate. In some embodiments, the alkylglycoside has a critical micelle concentration (CMC) of less than about 1 mM (e.g., 0.1 to 1 mM).

In some embodiments, the compositions may further comprise a mucosal delivery-enhancing agent selected from the group consisting of an aggregation inhibitory agent, a charge-modifying agent, a pH control agent, a degradative enzyme inhibitory agent, a mucolytic or mucus clearing agent, a chitosan, and a ciliostatic agent.

In some embodiments, the compositions may further comprise benzalkonium chloride or chloroethanol.

In some embodiments, the compositions may further comprise a membrane penetration-enhancing agent selected from the group consisting of a surfactant, a bile salt, a phospholipid additive, a mixed micelle, a liposome, a carrier, an alcohol, an enamine, a nitric oxide donor compound, a long-chain amphipathic molecule, a small hydrophobic penetration enhancer, a sodium or a salicylic acid derivative, a glycerol ester of acetoacetic acid, a cyclodextrin or beta-cyclodextrin derivative, a medium-chain fatty acid, a chelating agent, an amino acid or salt thereof, an N-acetylamino acid or salt thereof, an enzyme degradative to a selected membrane component and any combination thereof.

In some embodiments, pharmaceutical compositions are provided comprising at least one low molecular weight leptin related peptide of the present embodiments; a buffering agent; and an alkylglycoside, wherein the alkylglycoside is selected from the group consisting of dodecyl maltoside, tridecyl maltoside, sucrose mono-dodecanoate, sucrose mono-tridecanoate, and sucrose mono-tetradecanoate.

In some embodiments, there are provided formulations comprising at least one low molecular weight leptin related peptide, whether at high or low concentration, and at least one alkylglycoside and/or saccharide alkyl ester surfactant, hereinafter termed “alkylglycosides”. As used herein, “alkylglycoside” refers to any sugar joined by a linkage to any hydrophobic alkyl, as is known in the art. The linkage between the hydrophobic alkyl chain and the hydrophilic saccharide can include, among other possibilities, a glycosidic, ester, thioglycosidic, thioester, ether, amide or ureide bond or linkage. Examples of which are described herein. The terms alkylglycoside and alkylsaccharide may be used interchangeably herein.

In some embodiments, the alkylglycosides of the invention includes, but is not limited to, dodecyl maltoside, tridecyl maltoside, tetradecyl maltoside, sucrose mono-dodecanoate, sucrose mono-tridecanoate, and sucrose mono-tetradecanoate.

As used herein, a “surfactant” is a surface active agent which is agents that modify interfacial tension of water. Typically, surfactants have one lipophilic and one hydrophilic group in the molecule. Broadly, the group includes soaps, detergents, emulsifiers, dispersing and wetting agents, and several groups of antiseptics. More specifically, surfactants include stearyltriethanolamine, sodium lauryl sulfate, sodium taurocholate, laurylaminopropionic acid, lecithin, benzalkonium chloride, benzethonium chloride and glycerin monostearate; and hydrophilic polymers such as polyvinyl alcohol, polyvinylpyrrolidone, carboxymethylcellulose sodium, methylcellulose, hydroxymethylcellulose, hydroxyethylcellulose and hydroxypropylcellulose.

As used herein, “alkylglycoside” refers to any sugar joined by a linkage to any hydrophobic alkyl, as is known in the art. The hydrophobic alkyl can be chosen of any desired size, depending on the hydrophobicity desired and the hydrophilicity of the saccharide moiety. In one aspect, the range of alkyl chains is from 9 to 24 carbon atoms; and further the range is from 10 to 14 carbon atoms.

As used herein, “Critical Micelle Concentration” or “CMC” is the concentration of an amphiphilic component (alkylglycoside) in solution at which the formation of micelles (spherical micelles, round rods, lamellar structures etc.) in the solution is initiated. In some embodiments, the alkylglycoside has a critical micelle concentration (CMC) of less than about 1 mM (e.g., 0.1 to 1 mM) in pure water.

As used herein, “saccharide” is inclusive of monosaccharides, oligosaccharides or polysaccharides in straight chain or ring forms. Oligosaccharides are saccharides having two or more monosaccharide residues.

As used herein, “sucrose esters” are sucrose esters of fatty acids. Sucrose esters can take many forms because of the eight hydroxyl groups in sucrose available for reaction and the many fatty acid groups, from acetate on up to larger, more bulky fats that can be reacted with sucrose. This flexibility means that many products and functionalities can be tailored, based on the fatty acid moiety used. Sucrose esters have food and non-food uses, especially as surfactants and emulsifiers, with growing applications in pharmaceuticals, cosmetics, detergents and food additives. They are biodegradable, non-toxic and mild to the skin.

As used herein, a “suitable” alkylglycoside means one that fulfills the limiting characteristics of the invention, i.e., that the alkylglycoside be nontoxic and nonionic, and that it reduces the immunogenicity or aggregation of a low molecular weight leptin related peptide when it is administered via the ocular, nasal, nasolacrimal, sublingual, buccal, inhalation routes or by injection routes such as the subcutaneous, intramuscular, or intravenous routes. Suitable compounds can be determined using the methods set forth in the examples.

The compositions and formulations of the present invention may include a surfactant. The term “surfactant” comes from shortening the phrase “surface active agent”. In pharmaceutical applications, surfactants are useful in liquid pharmaceutical formulations in which they serve a number of purposes, acting as emulsifiers, solubilizers, and wetting agents. Emulsifiers stabilize the aqueous solutions of lipophilic or partially lipophilic substances. Solubilizers increase the solubility of components of pharmaceutical compositions increasing the concentration which can be achieved. A wetting agent is a chemical additive which reduces the surface tension of a fluid, inducing it to spread readily on a surface to which it is applied, thus causing even “wetting” of the surface with the fluids. Wetting agents provide a means for the liquid formulation to achieve intimate contact with the mucous membrane or other surface areas with which the pharmaceutical formulation comes in contact.

The surfactants of the invention can also include a saccharide. As use herein, a “saccharide” is inclusive of monosaccharides, oligosaccharides or polysaccharides in straight chain or ring forms, or a combination thereof to form a saccharide chain. Oligosaccharides are saccharides having two or more monosaccharide residues. The saccharide can be chosen, for example, from any currently commercially available saccharide species or can be synthesized. Some examples of the many possible saccharides to use include glucose, maltose, maltotriose, maltotetraose, sucrose and trehalose. Preferable saccharides include maltose, sucrose and glucose.

The surfactants of the invention can likewise consist of a sucrose ester. As used herein, “sucrose esters” are sucrose esters of fatty acids. Sucrose esters can take many forms because of the eight hydroxyl groups in sucrose available for reaction and the many fatty acid groups, from acetate on up to larger, more bulky fatty acids that can be reacted with sucrose. This flexibility means that many products and functionalities can be tailored, based on the fatty acid moiety used. Sucrose esters have food and non-food uses, especially as surfactants and emulsifiers, with growing applications in pharmaceuticals, cosmetics, detergents and food additives. They are biodegradable, non-toxic and mild to the skin.

While there are potentially many thousands of alkylglycosides which are synthetically accessible, the alkylglycosides dodecyl, tridecyl and tetradecyl maltoside and sucrose dodecanoate, tridecanoate, and tetradecanoate are particularly useful since they possess desirably low CMC's. Hence, the above examples are illustrative, but the list is not limited to that described herein. Derivatives of the above compounds which fit the criteria of the claims should also be considered when choosing a glycoside.

Examples from which useful alkylglycosides can be chosen for the therapeutic composition include: alkylglycosides, such as octyl-, nonyl-, decyl-, undecyl-, dodecyl-, tridecyl-, tetradecyl, pentadecyl-, hexadecyl-, heptadecyl-, and octadecyl-D-maltoside, -glucoside or -sucroside (i.e., sucrose ester) (synthesized according to Koeltzow and Urfer; Anatrace Inc., Maumee, Ohio; Calbiochem, San Diego, Calif.; Fluka Chemie, Switzerland); alkyl thiomaltosides, such as heptyl, octyl, dodecyl-, tridecyl-, and tetradecyl-.beta.-D-thiomaltoside (synthesized according to Defaye, J. and Pederson, C., “Hydrogen Fluoride, Solvent and Reagent for Carbohydrate Conversion Technology” in Carbohydrates as Organic Raw Materials, 247-265 (F. W. Lichtenthaler, ed.) VCH Publishers, New York (1991); Ferenci, T., J. Bacteriol, 144:7-11 (1980)); alkyl thioglucosides, such as heptyl- or octyl 1-thio .beta.- or .beta.-D-glucopyranoside (Anatrace, Inc., Maumee, Ohio; see Saito, S. and Tsuchiya, T. Chem. Pharm. Bull. 33:503-508 (1985)); alkyl thiosucroses (synthesized according to, for example, Binder, T. P. and Robyt, J. F., Carbohydr. Res. 140:9-20 (1985)); alkyl maltotriosides (synthesized according to Koeltzow and Urfer); long chain aliphatic carbonic acid amides of sucrose amino-alkyl ethers; (synthesized according to Austrian Patent 382,381 (1987); Chem. Abstr., 108:114719 (1988) and Gruber and Greber pp. 95-116); derivatives of palatinose and isomaltamine linked by amide linkage to an alkyl chain (synthesized according to Kunz, M., “Sucrose-based Hydrophilic Building Blocks as Intermediates for the Synthesis of Surfactants and Polymers” in Carbohydrates as Organic Raw Materials, 127-153); derivatives of isomaltamine linked by urea to an alkyl chain (synthesized according to Kunz); long chain aliphatic carbonic acid ureides of sucrose amino-alkyl ethers (synthesized according to Gruber and Greber, pp. 95-116); and long chain aliphatic carbonic acid amides of sucrose amino-alkyl ethers (synthesized according to Austrian Patent 382,381 (1987), Chem. Abstr., 108:114719 (1988) and Gruber and Greber, pp. 95-116).

Some preferred glycosides include maltose, sucrose, and glucose linked by glycosidic or ester linkage to an alkyl chain of 9, 10, 12, 13 or 14 carbon atoms, e.g., nonyl-, decyl-, dodecyl- and tetradecyl sucroside, glucoside, and maltoside. These compositions are nontoxic, since they are degraded to an alcohol or fatty acid and an oligosaccharide, and amphipathic.

In some embodiments, the compositions comprising at least one low molecular weight leptin related peptide may be prepared by admixing the peptide with a surfactant comprising of at least one alkylglycoside and/or sucrose ester, wherein the alkyl has from 10 to 14 carbon atoms.

In some embodiments, the compositions comprising at least one low molecular weight leptin related peptide may be prepared by admixing a leptin related peptide of the present embodiments, a stabilizing agent and a buffering agent, wherein the stabilizing agent is at least one alkylglycoside surfactant.

In some embodiments, the compositions comprising at least one low molecular weight leptin related peptide of the present invention may be used with a hydrogel, such as a absorption-enhancing self-assembling non-polymeric hydrogel. (See e.g., WO 2009/02954, incorporated herein by reference in its entirety.)

Other transmucosal absorption enhancers suitable for use in the present embodiments include, but are not limited to, chelators (e.g., EDTA, EGTA), non-ionic surfactants (e.g., 23-lauryl ether, laureth-9, polysorbates (including polysorbate 80), sucrose esters, or dodecylmaltoside), cationic surfactants (e.g., benzalkonium chloride or cetylmethylammonium bromide), anionic surfactants (e.g., sodium dodecyl glycocholate or sodium lauryl sulfate), bile salts and other steroidal detergents (e.g., cholate, deoxycholate, taurocholate, sodium glycocholate, sodium taurocholate, saponins, sodium taurodihydrofusidate or sodium glycodihydrofusidate), fatty acids (e.g., oleic acid, lauric acid capric acid, heptnoic acid, stearic acid, sucrose laurate, isopropyl myristate, sodium myristate or caprylic acid), and non-surfactants (e.g., aprotinin, dextran sulfate, sulfoxides, salicylates, Intravail®or 1-dodecylazacycloheptane-2-one(Azone)), phospholipids (e.g., phosphatidylcholines, lysophosphatidylcholine, or monoooleoyl phosphaltidyl ethanomamine), cyclodextrins, and various alkyl glycosides. In other embodiments, the transmucosal absorption enhancer can be benzalkonium chloride.

### Leptin

Leptin is the afferent signal in a negative feedback loop regulating food intake and body weight. The leptin receptor is a member of the cytokine receptor family. The anorexigenic effect of leptin is dependent on binding to homodimer of the Ob-R_{b} isoform of this receptor which encodes a long intracytoplasmic domain that includes several motifs for protein-protein interaction. Ob-R_{b} is highly expressed in the hypothalamus suggesting that this brain region is an important site of leptin action. Mutation of the mouse ob gene has been demonstrated to result in a syndrome that exhibits a pathophysiology that includes: obesity, increased body fat deposition, hyperglycemia, hyperinsulinemia, hypothermia, and impaired thyroid and reproductive functions in both male and female homozygous ob/ob obese mice. (See e.g., Ingalis, et al., J Hered 41: 317-318 (1950)). Therapeutic uses for leptin or leptin receptor include (i) diabetes (See, e.g., PCT Patent Applications WO98/55139, WO98/12224, and WO97/02004); (ii) hematopoiesis (See, e.g., PCT Patent Applications WO97/27286 and WO98/18486); (iii) infertility (See, e.g., PCT Patent Applications WO97/15322 and WO98/36763); and (iv) tumor suppression (See, e.g., PCT Patent Applications WO98/48831), each of which are incorporated herein by reference in their entirety.

The mature form of circulating leptin is a 146-amino acid protein that is normally excluded from the CNS by the blood-brain barrier (BBB) and the blood-CSF barrier. (See, e.g., Weigle et al., 1995. J Clin Invest 96: 2065-2070 (1995)). Leptin fragments, such as an 18 amino acid fragment comprising residues ⁵⁷VTGLDFIPGLHPILTLSK⁷⁴ (SEQ ID NO:19) taken from full length human leptin, SEQ ID NO:17, function in weight loss upon direct administration through an implanted cannula to the lateral brain ventricle of rats. (See, e.g., PCT Patent Applications WO97/46585, which is incorporated herein by reference in its entirety). However, the fragments in PCT Patent Applications WO97/46585 are different from the fragments of this invention. SEQ ID NO:17 is as follows:

(SEQ ID NO: 17)
MHWGTLCGFLWLWPYLFYVQ AVPIQKVQDDTKTLIKTIVT
RINDISHTQSVSSKQKVTGL DFIPGLHPILTLSKMDQTLA
VYQQILTSMPSRNVIQISND LENLRDLLHVLAFSKSCHLP
WASGLETLDSLGGVLEASGY STEVVALSRLQGSLQDMLWQ LDLSPGC

SEQ ID NO:2 and 18, which depict mouse and human OB3, respectively, as well as various fragments, derivatives, analogs and homologs thereof, are low molecular weight leptin-related peptides comprising the C-terminal amino acid residues 116-122 of native leptin (LEP) (the full length mouse and human leptin proteins are depicted in SEQ ID NOS:1 and 17, respectively). As used herein, the LEP(116-122) peptide is hereforth referred to as “OB3.” The various low molecular weight leptin-related peptides of the invention are:

Leptin Peptides - Single Letter Amino Acid
Codes
(SEQ ID NO: 2)

| | |
|---|---|
| (i) | S C S L P Q T; |

(SEQ ID NO: 3)

| | |
|---|---|
| (ii) | A V P I Q K V Q D D T K T L I; |

(SEQ ID NO: 4)

| | |
|---|---|
| (iii) | T K T L I K T I V T R I N D I; |

(SEQ ID NO: 5)

| | |
|---|---|
| (iv) | R I N D I S H T Q S V S A K Q; |

(SEQ ID NO: 6)

| | |
|---|---|
| (v) | V S A K Q R V T G L D F I P G; |

(SEQ ID NO: 7)

| | |
|---|---|
| (vi) | D F I P G L H P I L S L S K M; |

(SEQ ID NO: 8)

| | |
|---|---|
| (vii) | S L S K M D Q T L A V Y Q Q V; |

(SEQ ID NO: 9)

| | |
|---|---|
| (viii) | V Y Q Q V L T S L P S Q N V L; |

(SEQ ID NO: 10)

| | |
|---|---|
| (ix) | S Q N V L Q I A N D L E N L R; |

(SEQ ID NO: 11)

| | |
|---|---|
| (x) | D L L H L L A F S K S C S L P; |

(SEQ ID NO: 12)

| | |
|---|---|
| (xi) | S C S L P Q T S G L Q K P E S; |

(SEQ ID NO: 13)

| | |
|---|---|
| (xii) | Q K P E S L D G V L E A S L Y; |

(SEQ ID NO: 14)

| | |
|---|---|
| (xiii) | E A S L Y S T E V V A L S R L; |

(SEQ ID NO: 15)

| | |
|---|---|
| (xiv) | A L S R L Q G S L Q D I L Q Q; |

(SEQ ID NO: 16)

| | |
|---|---|
| (xv) | D I L Q Q L D V S P E C; |
| and | |

(SEQ ID NO: 18)

| | |
|---|---|
| (xvi) | S C H L P W A |

OB3 possesses the ability to modulate body mass homeostasis in test animals upon i.p. (intraperitoneal) administration. OB3 polypeptides of the invention include peptides composed of all L-isoform amino acids, all D-isoform amino acids, as well as variants containing both L-isoform and D-isoform amino acids. By way of non-limiting example, specific mouse D-substituted OB3 peptides of SEQ ID NO:2 include:

| | |
|---|---|
| [D-Ser-1]-OB3, | (SEQ ID NO: 21) |
| [D-Cys-2]-OB3, | (SEQ ID NO: 22) |
| [D-Ser-3]-OB3, | (SEQ ID NO: 23) |
| [D-Leu-4]-OB3, | (SEQ ID NO: 24) |
| [D-Pro-5]-OB3, | (SEQ ID NO: 25) |
| [D-Gln-6]-OB3, | (SEQ ID NO: 26) |
| [D-Thr-7]-OB3, | (SEQ ID NO: 27) |
| and | |
| All [D]-OB3. | (SEQ ID NO: 28) |

Similarly, specific human D-substituted OB3 peptides of SEQ ID NO:18 include, but are not limited to:

| | |
|---|---|
| [D-Ser-1]-OB3, | (SEQ ID NO: 29) |
| [D-Cys-2]-OB3, | (SEQ ID NO: 30) |
| [D-His-3]-OB3, | (SEQ ID NO: 31) |
| [D-Leu-4]-OB3, | (SEQ ID NO: 32) |
| [D-Pro-5]-OB3, | (SEQ ID NO: 33) |
| [D-Trp-6]-OB3, | (SEQ ID NO: 34) |
| [D-Ala-7]-OB3, | (SEQ ID NO: 35) |
| and | |
| all [D]-OB3, | (SEQ ID NO: 36) |

One preferred D-substituted OB3 peptide is the mouse or human [D-Leu-4]-OB3 peptide (SEQ ID NOS: 24 or 32, respectively). In addition, OB3 peptides of the invention may contain D-substituted amino acids for any two, three, four, five, or six positions. For example, one di-D-amino acid substituted OB3 peptide is [D-Leu-4, D-Pro-5]-OB3 (SEQ ID NO:37).

Also disclosed herein are leptin-related peptides comprising N-terminal amino acids 21-35, 31-45, 41-55 and 51-65 of native leptin, and hereforth referred to as LEP(21-35) (SEQ ID NO:3), LEP(31-45) (SEQ ID NO:4), LEP(41-55) (SEQ ID NO:5) and LEP(51-65) (SEQ ID NO:6), respectively, and fragments, derivatives, analogs and homologs thereof.

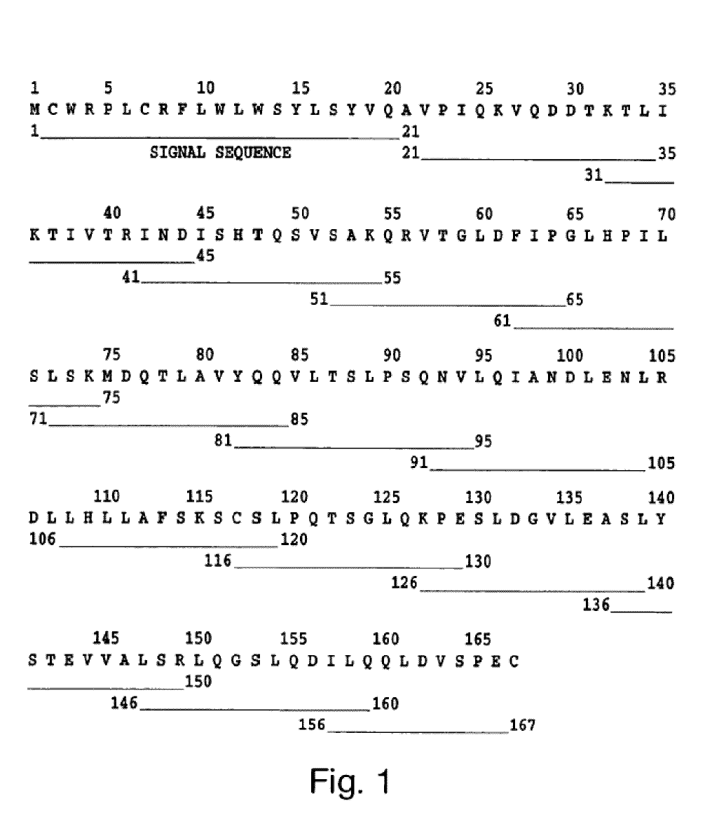

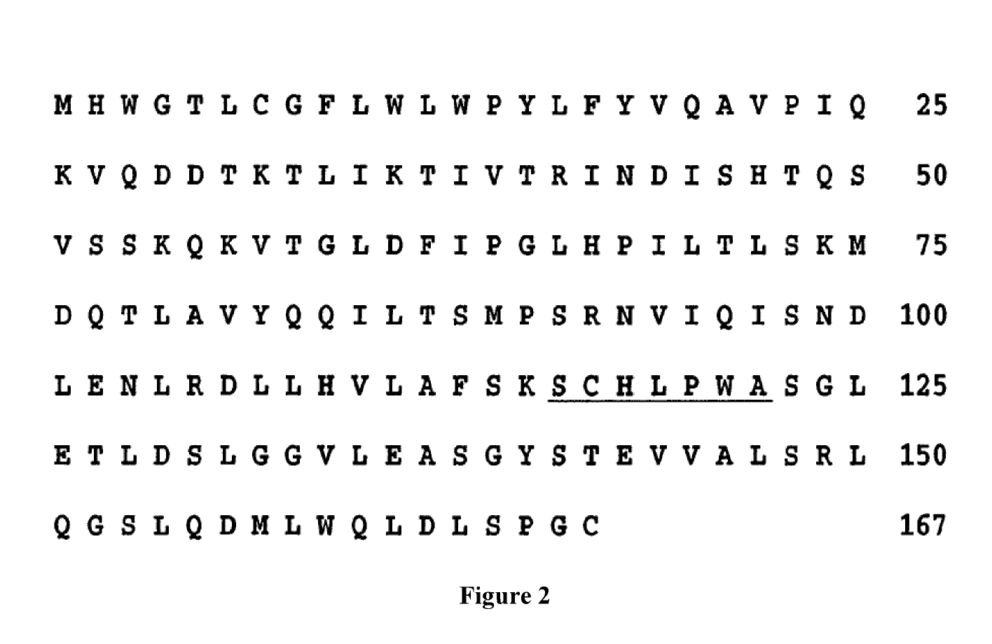

Additional peptides of the invention include, for example, LEP(61-75) (SEQ ID NO:7), LEP(71-85) (SEQ ID NO:8), LEP(81-95) (SEQ ID NO:9), LEP(91-105) (SEQ ID NO:10), LEP(106-120) (SEQ ID NO:11), LEP(116-130) (SEQ ID NO:12), LEP(126-140) (SEQ ID NO:13), LEP(136-150) (SEQ ID NO:14), LEP(146-160) (SEQ ID NO:15), and LEP(156-167) (SEQ ID NO:16). See, e.g., FIG. 1 and FIG. 2 for mouse and human full length protein, respectively. Any of the OB3 and OB3-related peptides of the present invention, as well as the D-isoforms, fragments, derivatives, analogs, and homologs thereof, are exceptionally strong candidates for the development of leptin-related analogs, or mimetics.

According to some embodiments, the low molecular weight leptin related peptides, or OB3 polypeptides, comprise the amino acid sequence of any one of SEQ ID NO: 2-16, 18, and 19 or one of the related D amino acid variants referred herein as SEQ ID NO: 20-37.

The low molecular weight leptin related peptides may be 6 to 25 amino acids in length and comprise the amino acid sequence of any one of SEQ ID NO: 2-16, 18, and 19 or one of the related D amino acid variants referred herein as SEQ ID NO: 20-37, as appropriate. In some embodiments, the low molecular weight leptin related peptides are from 6 to 15 amino acids in length. The above ranges are inclusive of narrower ranges contained within and each of the specific examples are meant to be representative of the broader range. Examples of the narrower ranges include, but are not limited to, 6 to 7, 6 to 9, 6 to 12, 6 to 15, 6 to 18, 6 to 20, 6 to 25, 7 to 9, 7 to 12, 7 to 13, 7 to 15, 7 to 18, 7 to 20, 7 to 25, 9 to 12, 9 to 15, 9 to 18, 9 to 20, 9 to 25, 10 to 15, 10 to 18, 10 to 20, 10 to 25, 12 to 15, 12 to 18, 12 to 20, 12 to 25, 15 to 18, 15 to 20, 15 to 25, 15 to 18, 15 to 20, and 15 to 25 amino acids in length.

In some embodiments, the low molecular weight leptin related peptides and related peptidic compounds have the formula X1-C—X2, where C comprises the amino acid sequence of any one of SEQ ID NO: 2-16 and 18-37, where X1 and X2 are each 0 to 19 amino acids in length, with the proviso that the total length of the peptide is no more than 25 amino acids. For example, where a low molecular weight leptin related peptides is 7 amino acid in length, as with SEQ ID NO: 18, the amino acid sequence of SEQ ID NO: 8 may be contained in larger amino acid sequence such as a peptide of 8 to 25 amino acids. In such an instance, the amino acid sequence of SEQ ID NO: 18 is referred to as the core sequence. A low molecular weight leptin related peptides comprising SEQ ID NO: 18 may therefore be represented by the following formula: X1-C—X2, where X1 and X2 are each 0 to 19 amino acids in length, wherein the total length of the peptide is no more than 25 amino acids. Accordingly, the maximum value of the sum of X1 and X2 may be determined by subtracting the length of the core sequence from the total length of the low molecular weight leptin related peptides. In preferred embodiments, the maximum value of the sum of X1 and X2 is selected from the group consisting of 0, 1, 2, 3, 4, 5, 6, 7, 8, 9, 10, 11, 12, 13, 14, 15, 16, 17, 18, or 19.

According to some embodiments, the low molecular weight leptin related peptides and peptidic compounds are 6 to 25 amino acids in length and includes at least 6, 7, 8, 9, 10, 11, 12, or 13 amino acids from any one of the amino acid sequences of SEQ ID NO: 2-16 and 18-37, as appropriate, wherein the at least 6, 7, 8, 9, 10, 11, 12, or 13 amino acids maintain their relative positions as they appear in the amino acid sequences of SEQ ID NO: 2-16 and 18-37. In some embodiments, the at least 6, 7, 8, 9, 10, 11, 12, or 13 amino acids maintain their relative positions within the original length of the core sequence of one of SEQ ID NO: 2-16 and 18-37.

According to some embodiments, the low molecular weight leptin related peptides and peptidic compounds include at least 6, 7, 8, 9, 10, 11, 12, or 13 consecutive amino acids of any one of the amino acid sequences of SEQ ID NO: 2-16 and 18-37 and consist of between 6 and 25 amino acids, inclusive.

In some embodiments, the core sequence of the low molecular weight leptin related peptides or peptidic compounds has an amino acid sequence that is at least 60, 70, 80, 85, 90, 95, 98, 99, or 100% identical to any one of SEQ ID NO: 2-16 and 18-37.

In some embodiments, there is provided low molecular weight leptin related peptides and related peptidic compounds that comprise variants of the core sequence (C). In these embodiments, the low molecular weight leptin related peptides are 6 to 25 amino acids long comprising the amino acid sequence of any one of SEQ ID NO: 2-16 and 18-37, as above, wherein one, two, three, or four amino acids have been substituted, deleted from, and/or inserted into the core amino acid sequence. In some embodiments, the alanine substitutions at one or more of amino acid positions may be used. Other preferred substitutions include conservative substitutions that have little or no effect on the overall net charge, polarity, or hydrophobicity of the protein. Conservative substitutions are set forth in the table below.

### Conservative Amino Acid Substitutions

| | |
|---|---|
| Basic: | arginine |
| | lysine |
| | histidine |
| Acidic: | glutamic acid |
| | aspartic acid |
| Uncharged Polar: | glutamine |
| | asparagine |
| | serine |
| | threonine |
| | tyrosine |
| Non-Polar: | phenylalanine |
| | tryptophan |
| | cysteine |
| | glycine |
| | alanine |
| | valine |
| | praline |
| | methionine |
| | leucine |
| | isoleucine |

The table below sets out another scheme of amino acid substitution:

| Original Residue | Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr; Ile |
| Phe | Met; Leu; Tyr |
| Ser | Thr |
| Thr | Ser |
| Trp | Tyr |
| Tyr | Trp; Phe |
| Val | Ile; Leu |

Other substitutions can consist of less conservative amino acid substitutions, such as selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to have a more significant effect on function are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine.

### Isolation of Homologs

Oligonucleotide probe or probes may be designed to correspond to sequences known for a particular clone. This sequence can be derived from the sequences provided herein, or from a combination of those sequences.

Homologs (i.e., nucleic acids encoding the aforementioned peptides derived from species other than human) or other related sequences (e.g., paralogs) can also be obtained by low, moderate or high stringency hybridization with all or a portion of the particular human sequence as a probe using methods well known in the art for nucleic acid hybridization and cloning.

A nucleic acid sequence that is hybridizable to a nucleic acid sequence (or a complement of the foregoing) encoding the aforementioned peptides, or a derivative of the same, under conditions of high stringency is provided. By way of example and not limitation, procedures using such conditions of high stringency are as follows: Step 1: Filters containing DNA are pretreated for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Step 2: Filters are hybridized for 48 hours at 65° C. in the above prehybridization mixture to which is added 100 mg/ml denatured salmon sperm DNA and 5-20×10⁶ cpm of ³²P-labeled probe. Step 3: Filters are washed for 1 hour at 37° C. in a solution containing 2×SSC, 0.01% PVP, 0.01% Ficoll, and 0.01% BSA. This is followed by a wash in 0.1×SSC at 50° C. for 45 minutes. Step 4: Filters are autoradiographed. Other conditions of high stringency that may be used are well known in the art. (See, e.g., Ausubel et al., (eds.), 1993, CURRENT PROTOCOLS IN MOLECULAR BIOLOGY, John Wiley and Sons, NY; and Kriegler, 1990, GENE TRANSFER AND EXPRESSION, A LABORATORY MANUAL, Stockton Press, NY).

A nucleic acid sequence that is hybridizable to a nucleic acid sequence (or a complement of the foregoing) encoding the aforementioned peptides, or a derivatives, under conditions of moderate stringency is also provided. By way of example and not limitation, procedures using such conditions of moderate stringency are as follows: Step 1: Filters containing DNA are pretreated for 6 hours at 55° C. in a solution containing 6×SSC, 5×Denhardt's solution, 0.5% SDS and 100 mg/ml denatured salmon sperm DNA. Step 2: Filters are hybridized for 18-20 hours at 55° C. in the same solution with 5-20×106 cpm ³²P-labeled probe added. Step 3: Filters are washed at 37° C. for 1 hour in a solution containing 2×SSC, 0.1% SDS, then washed twice for 30 minutes at 60° C. in a solution containing 1×SSC and 0.1% SDS. Step 4: Filters are blotted dry and exposed for autoradiography. Other conditions of moderate stringency that may be used are well-known in the art. (See, e.g., Ausubel et al., (eds.), 1993, CURRENT PROTOCOLS IN MOLECULAR BIOLOGY, John Wiley and Sons, NY; and Kriegler, 1990, GENE TRANSFER AND EXPRESSION, A LABORATORY MANUAL, Stockton Press, NY).

A nucleic acid that is hybridizable to a nucleic acid sequence disclosed in this invention or to a nucleic acid sequence encoding a the aforementioned peptides, or fragments, analogs or derivatives under conditions of low stringency, is further provided. By way of example and not limitation, procedures using such conditions of low stringency are as follows (See also Shilo and Weinberg, 1981, Proc Natl Acad Sci USA 78: 6789-6792): Step 1: Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Step 2: Filters are hybridized for 18-20 hours at 40° C. in the same solution with the addition of 0.02% PVP, 0.02% Ficoll, 0.2% BSA, 100 μg/ml salmon sperm DNA, 10% (wt/vol) dextran sulfate, and 5-20×106 cpm ³²P-labeled probe. Step 3: Filters are washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Step 4: Filters are blotted dry and exposed for autoradiography. If necessary, filters are washed for a third time at 65-68° C. and reexposed to film. Other conditions of low stringency that may be used are well known in the art (e.g., as employed for cross-species hybridizations). (See, e.g., Ausubel et al., (eds.), 1993, CURRENT PROTOCOLS IN MOLECULAR BIOLOGY, John Wiley and Sons, NY; and Kriegler, 1990, GENE TRANSFER AND EXPRESSION, A LABORATORY MANUAL, Stockton Press, NY).

The invention also relates to nucleic acids hybridizable to or complementary to the foregoing sequences, in particular the invention provides the inverse complement to nucleic acids hybridizable to the foregoing sequences (i.e., the inverse complement of a nucleic acid strand has the complementary sequence running in reverse orientation to the strand so that the inverse complement would hybridize with little or no mismatches to the nucleic acid strand). In specific aspects, nucleic acid molecules encoding derivatives and analogs of an aforementioned peptide (supra), or antisense nucleic acids to the same (See, e.g., infra) are additionally provided.

### Derivatives of OB3: Truncated OB3 and D-Amino Acid Substituted OB3

To date, four general classes of anti-obesity drugs have been developed. These pharmacophores are designed to induce a state of negative energy balance, i.e., a state where energy expenditure exceeds energy intake, thus resulting in weight loss, through a number of different mechanisms. LEP-(116-130) (SEQ ID NO:2) effects on energy balance and glucose homeostasis do not require peptide activation of the long form of the leptin receptor. (See Grasso et al., Diabetes 48:2204-2209 (1999) and Grasso et al., Regulatory Peptides 85(23):93-100 (1999)). Amino acid residues 116-122 (OB3) of mouse leptin have the minimal active epitope in this region of the molecule, and the potency of OB3 can be increased by inversion of the configuration of the L-leucine residue at position 4 by substitution with its D-isoform. (See U.S. Pat. No. 6,777,388, U.S. Pat. No. 7,208,572, and U.S. Pat. No. 7,186,694).

LEP-(116-130) is a synthetic peptide that has been shown to regulate energy balance and blood glucose levels in ob/ob and db/db mice (see Grasso et al., Endocrinology 138(4):1413-1418 (1997); Grasso et al., Diabetes 48:2204-2209 (1999) and Grasso et al., Regulatory Peptides 85(23):93-100 (1999)), stimulate prolactin and luteinizing hormone secretion in male rats (see Gonzalez et al., Neuroendocrinology 70:213-220 (1999)), and enhance proliferative activity in rat adrenal cortex (see Malendowics et al., Medical Science Research 27:675-676 (1999)). A truncation strategy was used to demonstrate that the active epitope in LEP-(116-130) is composed of amino acid residues 116-122, i.e. the synthetic peptide amide corresponding to this epitope is OB3. Single-point D-amino acid substitution was used to study the structure-function relationships of each amino acid residue in OB3, and to increase its efficacy. The restricted domain represented by OB3 contains a functional epitope, which has the ability to mimic at least some of the effects of leptin on energy balance and glucose homeostasis.

The design of peptide ligands involved the introduction of conformational constraints into native sequences by techniques that include, but are not limited to, D-amino acid substitution or cyclization. (Hruby and Bonner, Methods Mol Biol. 35:201-40 (1994)). Systematic replacement of L-amino acids by their D-amino acid isoforms was used to determine the stereostructural requirements of specific residues in a peptide for peptide-receptor interaction, and to assess the contribution of certain secondary structural motifs, e.g., α-helix or β-turn, to the bioactivity of the peptide. (Hruby, Biopolymers 33(7):1073-82 (1993) and Hruby, Life Sci. 52(10):845-55 (1993)). This approach increased peptide resistance to enzymatic hydrolysis, and to enhance the properties of biologically active peptides, including receptor binding, functional potency, and duration of action. (See Fauchere et al., Adv. Drug Res., 23:127-139 (1992); Doherty et al., J. Med. Chem., 36:2585-2594 (1993); Kirby et al., J. Med. Chem., 36:3802-3808 (1993); Morita et al., FEBS Lett., 353:84-88 (1994)).

The computer program ChemSite was used to construct molecular models of OB3 as well as its D-amino acid-substituted analogs, and to measure their surface areas. Introducing conformational constraints into OB3 via D-amino acid substitution resulted in a molecular configuration favoring protection of critical peptide bonds from enzymatic hydrolysis, which accounted for the increased potency of [D-Leu-4]-OB3.

Of the eight D-amino acid-substituted peptide analogs tested in this study, [D-Leu-4]-OB3 was more potent (2.6-fold) in reducing body weight gain than native OB3. This analog also had greater anorexigenic activity than OB3, and significantly reduced water intake. The most striking action of [D-Leu-4]-OB3, however, was related to its effects on blood glucose. In contrast to OB3, which maximally reduced blood glucose levels by approximately 100 mg/dl, [D-Leu-4]-OB3 lowered blood glucose to levels seen in nondiabetic wild type mice within two days of peptide treatment. The effects of [D-Leu-4]-OB3 on blood glucose are physiologically related to the reduced water consumption observed in mice treated with this peptide via its ability to decrease the polyuria associated with hyperglycemia.

A similar correlation between blood glucose levels and water intake was observed in mice treated with [D-Pro-5]-OB3, although its antihyperglycemic effect occurred with a different time course, i.e., after four days of peptide treatment. Moreover, [D-Leu-4]-OB3 also increases tissue sensitivity to insulin (see Grasso et al., Regu. Pept., 101: 123-129 (2001)), and suggests a possible role for leptin-related peptides in the treatment of type 2 diabetes.

Because [D-Leu-4]-OB3 and [D-Pro-5]-OB3 appeared to be more effective than the other D-amino acid-substituted analogs, as compared to OB3, in most of the parameters measured, these results suggest that OB3 contains a sequence that is highly sensitive to changes in stereochemical configuration.

Utilizing a truncation strategy, it was demonstrated that the activity of LEP-(116-130) resides in a restricted domain between amino acid residues 116-122. The synthetic peptide representing this region has been named OB3. D-amino acid substitution was used to determine the stereospecificity of each residue in OB3, and to create a more potent analog of OB3, [D-Leu-4]-OB3. Synthetic peptide strategies are useful in the development of potent and stabile pharmacophores with potential therapeutic significance in the treatment of human obesity and its related metabolic dysfunctions, including, for example, type 2 diabetes.

### Leptin-Related Peptides, and Derivatives, Fragments, Homologs and Analogs Thereof

In one embodiment, the present invention relates to methods of utilizing leptin-related peptides to increase bone formation. Preferablly the leptin-related peptides are related to an animal leptin, particularly mammalian leptin, or most particularly, a human leptin. These peptides may also be synthesized peptides. In one embodiment, the peptide is chosen from the C-terminal portion of the leptin protein, while in another embodiment, the peptide is chosen from the N-terminal portion of the leptin protein. The present invention also relates utilizing derivatives, fragments, homologs, analogs and variants of the aforementioned peptides. The peptides utilized in this invention can also include fusion proteins, particularly where the peptide is fused to a protein selected from the group consisting of alkaline phosphatase, glutathione-S-transferase and green fluorescent protein, or any antibody tag known in the art including myc 9E10, His tag, flag tag, and the like.

The present invention additionally relates to nucleic acids that encode the leptin-related peptides of the claimed invention. Specifically, the nucleic acids provided, comprise the coding regions, non-coding regions, or both, either alone or cloned in a recombinant vector, as well as oligonucleotides and related primer and primer pairs corresponding thereto. The nucleic acid strand may also be the complementary nucleic acid strand. Nucleic acids may be DNA, RNA, or a combination thereof. The vectors of the invention may be expression vectors.

Nucleic acids encoding said peptides may be obtained by any method known within the art (e.g., by PCR amplification using synthetic primers hybridizable to the 3′- and 5′-termini of the sequence and/or by cloning from a cDNA or genomic library using an oligonucleotide sequence specific for the given gene sequence, or the like).

In one embodiment, a leptin peptide utilized may have an amino acid sequence Xaaₙ-Ser-Cys-Xaa₁-Leu-Pro-Xaa₂-Xaa₃-Xaaₙ, (SEQ ID NO:20) wherein Xaaₙ may be zero residues in length, or may be a contiguous stretch of peptide residues derived from SEQ ID NOS: 1 or 17, preferably a stretch of between 1 and 7 at either the C-terminus or N-terminus, most preferably the peptide is a total of 15 amino acids or less in length. In another embodiment, Xaa₁, Xaa₂ or Xaa₃ may be any amino acid substitution. In yet another embodiment, Xaa₁, Xaa₂ or Xaa₃ may be any conservative amino acid substitution of the respective residues in full length mouse or human leptin (SEQ ID NOS:1 and 17, respectively).

In further embodiments, Xaa₁ may be selected from the group consisting of His or Ser, and Xaa₂ or Xaa₃ may be any amino acid substitution. In another embodiment, Xaa₂ may be selected from the group consisting of Trp or Gln, and Xaa₁ or Xaa₃ may any amino acid substitution. In yet another embodiment, Xaa₃ may be selected from the group consisting of Ala or Thr, and Xaa₁ or Xaa₂ may be any amino acid substitution. In a preferred embodiment, Xaa₁ may be selected from the group consisting of His or Ser, Xaa₂ may be selected from the group consisting of Trp or Gln, and Xaa₃ is selected from the group consisting of Ala or Thr.

Species homologs of the disclosed polynucleotides and peptides are also provided by the present invention.

### Isolated Peptides and Polynucleotides

GenBank Accession numbers for mouse and human leptin and leptin receptor, providing the nucleotide and amino acid sequences for the disclosed leptin-related peptides and their encoding nucleic acids of the present invention, are GenBank Accession No. AF098792, GenBank Accession No. U22421, and GenBank Accession No. NM_{—}000230. Those skilled in the art will recognize that the predicted amino acid sequence can be determined from its nucleotide sequence using standard protocols well known in the art. The amino acid sequence of the peptide encoded by a particular clone is also be determined by expression of the clone in a suitable host cell, collecting the peptide and determining its sequence.

The peptides utilized by the methods disclosed herein also encompass allelic variants of the disclosed polynucleotides or peptides; that is, naturally-occurring alternative forms of the isolated polynucleotide which also encode peptides which are identical, homologous or related to those encoded by the polynucleotides. Alternatively, non-naturally occurring variants may be produced by mutagenesis techniques and/or by direct synthesis.

Derivatives, fragments, and analogs provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively. Fragments are, at most, one nucleic acid-less or one amino acid-less than the wild type full length sequence. Derivatives and analogs may be full length or other than full length, if said derivative or analog contains a modified nucleic acid or amino acid, as described infra. Derivatives or analogs of the aforementioned peptides include, but are not limited to, molecules comprising regions that are substantially homologous to the aforementioned peptides, in various embodiments, by at least about 30%, 50%, 70%, 80%, or 95% identity (with a preferred identity of 80-95%) over an amino acid sequence of identical size or when compared to an aligned sequence in which the alignment is done by a computer homology program known in the art, or whose encoding nucleic acid is capable of hybridizing to the complement (e.g., the inverse complement) of a sequence encoding the aforementioned peptides under stringent (the preferred embodiment), moderately stringent, or low stringent conditions. (See e.g., Ausubel, et al., CURRENT PROTOCOLS IN MOLECULAR BIOLOGY, John Wiley and Sons, New York, N.Y., 1993), and infra.

The peptides utilized by the present invention are functionally active. The aforementioned peptides, and fragments, derivatives, homologs or analogs thereof, are related to animals (e.g., mouse, rat, pig, cow, dog, monkey, frog), insects (e.g., fly), plants or, most preferably, human leptin. As used herein, the term “functionally active” refers to species displaying one or more known functional attributes of a full-length leptin. The peptides utilized herein also have the ability to cross the blood-brain barrier.

### Therapeutic Uses and Biological Activity

The polynucleotides and peptides of the present invention are expected to exhibit one or more of the uses or biological activities (including those associated with assays cited herein) identified below. Uses or activities described for peptides of the present invention may be provided by administration or use of such peptides or by administration or use of polynucleotides encoding such peptides (such as, for example, in gene therapies or vectors suitable for introduction of DNA).

### Research Uses and Utilities

The polynucleotides provided by the present invention can be used by the research community for various purposes. The polynucleotides can be used to express recombinant peptides for analysis, characterization or therapeutic use; as markers for tissues in which the corresponding peptides is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-peptide antibodies using DNA immunization techniques; and as an antigen to raise anti-DNA antibodies or elicit another immune response. Where the polynucleotide encodes a peptides which binds or potentially binds to another protein (such as, for example, in a receptor-ligand interaction), the polynucleotide can also be used in interaction trap assays (such as, for example, that described in Gyuris et al., Cell 75: 791-803 (1993)) to identify polynucleotides encoding the other protein or receptor with which binding occurs or to identify inhibitors of the binding interaction.

The peptides provided by the present invention can similarly be used in assay to determine biological activity, including in a panel of multiple peptides for high-throughput screening; to raise antibodies or to elicit another immune response; as a reagent (including the labeled reagent) in assays designed to quantitatively determine levels of the peptides (or its receptor) in biological fluids; as markers for tissues in which the corresponding peptides is most biologically active (either constitutively or at a particular stage of tissue differentiation or development or in a disease state); and, of course, to isolate correlative receptors. Where the peptide binds or potentially binds to another protein (such as, for example, in a receptor-ligand interaction), the peptide can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation: “MOLECULAR CLONING: A LABORATORY MANUAL”, 2d ed., Cold Spring Harbor Laboratory Press, Sambrook et al. (eds.), 1989; and “METHODS IN ENZYMOLOGY (Vol. 152): Guide to Molecular Cloning Techniques”, Academic Press, Berger and Kimmel (eds.), 1987.

### Utility for OB3 and Leptin-Related Peptides of the Invention

OB3 and leptin-related peptides of the invention are exceptionally strong candidates for the development of leptin-related analogs, or mimetics, with potential application to treatment of human pathophysiologies related to body weight homeostasis. Serum osteocalcin levels in ob/ob mice were lower than those seen in their sex- and age-matched nonobese counterparts. Mouse [D-Leu-4]-OB3 significantly elevated serum osteocalcin to levels higher than those seen in wild type control mice. OB3 and leptin-related peptides of the invention have greatly improved efficacy of treatment over recombinant leptin protein. The increased efficacy is due in part to the increased ability of these peptides to cross the blood brain barrier. Additional mechanism include their interaction with receptors other than the previously identified OB-R receptor encoded by the db gene.

The present invention provides a method for treatment or prevention of various diseases and disorders by administration of a biologically-active therapeutic compound (hereinafter “Therapeutic”). Such Therapeutics include but are not limited to: (i) any one or more of the aforementioned peptides, and derivative, fragments, analogs and homologs thereof; (ii) antibodies directed against the aforementioned peptides; (iii) nucleic acids encoding an aforementioned peptide, and derivatives, fragments, analogs and homologs thereof; (iv) antisense nucleic acids to sequences encoding an aforementioned peptide, and (v) modulators (i.e., inhibitors, agonists and antagonists).

Diseases or disorders associated with levels of activity or aberrant levels of the aforementioned peptides may be treated by administration of a Therapeutic that modulates activity.

### Disorders

Diseases and disorders that are characterized by increased (relative to a subject not suffering from said disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to, (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) are utilized to “knockout” endogenous function of an aforementioned peptide by homologous recombination (See, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from said disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

In a given embodiment, antibodies for the aforementioned peptides, or derivatives, fragments, analogs or homologs thereof that contain the antibody derived binding domain, are utilized as pharmacologically active compounds (hereinafter “Therapeutics”).

### Use of Leptin-Related Peptide to Treat Wasting Diseases

Mouse [D-Leu-4]-OB3 delivered in Intravail® is orally active; and demonstrates high bioavailability when compared to commonly used injection methods of administration; and exerts significant pharmacodynamic effects on body weight gain, food intake, serum glucose and osteocalcin levels. Thus, the potential for therapeutic application of [D-Leu-4]-OB3 extends not only to the treatment of obesity, but also to diabetes, anorexia nervosa, osteoporsis, cancer, as well as other wasting diseases.

The appearance of a biphasic absorption profile associated with intranasal delivery of mouse [D-Leu-4]-OB3 was observed with oral administration of mouse [D-Leu-4]-OB3 that was not observed in the absorption profiles associated with ip, subcutaneous (sc), or intramuscular (im) administration. The time course of this profile suggested a two-compartment model of peptide distribution in which the early peak may represent a very rapid systemic uptake of mouse [D-Leu-4]-OB3 across the nasal mucosa, and the later peak much slower gastrointestinal absorption. The studies also show that gastrointestinal absorption of mouse [D-Leu-4]-OB3 does occur, and that its bioavailability is significantly improved by Intravail®. Further, it is demonstrated in the examples herein that mouse [D-Leu-4]-OB3 retains bioactivity when given orally by gavage, and describe its effects on energy balance, glycemic control, and serum osteocalcin levels in wild type and genetically obese C57BL/6J ob/ob mice.

Those skilled in the art will recognize that mouse [D-Leu-4]-OB3 is a small peptide amide seven amino acids in length, relatively inexpensive to produce commercially, and does not require a saturable transport system for passage across the BBB. Because most cases of human obesity are characterized by leptin resistance due to defective transport across the BBB, this last characteristic makes [D-Leu-4]-OB3 especially attractive for potential treatment of human obesity and its related metabolic dysfunctions. Moreover, no obvious toxic side effects have ever been observed in mice or rats treated with [D-Leu-4]-OB3, or any of its bioactive analogs or homologs.

Oral or intranasal delivery of mouse [D-Leu-4]-OB3 provides non-invasive and convenient drug delivery, that not only reduces the discomfort and risk of infection associated with injection methods, but also fosters higher levels of patient compliance.

Results of earlier preclinical studies with mouse [D-Leu-4]-OB3 (See U.S. Pat. Nos. 6,777,388; 7,186,694; 7,208,572B2; Australian Patent number 772,278), have shown that intraperitoneal (ip) delivery of this peptide significantly improves a number of metabolic dysfunctions associated with the obesity syndrome in the ob/ob mouse model. (See Rozhayskaya-Arena M, et al., Endocrinology 141:2501-2517 (2000); Grasso P et al., Regulatory Pep. 101:123-129 (2001)).

Recently, studies have shown that intranasal delivery of mouse [D-Leu-4]-OB3 in Intravail® (Aegis Therapeutics, San Diego, Calif.), a patented transmucosal absorption enhancing agent, results in significantly higher bioavailability of mouse [D-Leu-4]-OB3 when compared to ip and other commonly used injection methods of drug delivery. (See Novakovic Z M et al., Regulatory Peptides 154:107-111 (2009)).

The availability of a new class of patented alkylsaccharide transmucosal absorption enhancing agents, collectively known as Intravail® (Aegis Therapeutics, San Diego, Calif.), has provided opportunities for the design of new therapeutic approaches to the delivery of protein and peptide drugs usually administered by injection. The chemistry, metabolism, and mechanisms by which Intravail® enhances transmucosal absorption have been previously discussed. (See Maggio E T. Expert Opin Drug Deliv 3:529-539 (2006)).

Although a number of naturally occurring peptides, including but not limited to, insulin, glucagon, erythropoietin, calcitonin, parathyroid hormone, and growth hormone, have therapeutic application to the treatment of disease, the inherent susceptibility of these proteins and peptides to aggregation, denaturation, proteolytic hydrolysis, and/or poor absorption from the gastrointestinal tract thus far has made them unlikely candidates for oral delivery.

Reformulation of a number of protein and peptide drugs with transmucosal absorption agents, including arginine vasopressin (see Maggio E T. Expert Opin Drug Deliv 3:529-539 (2006)); calcitonin (see Ahsan F et al., Pharm. Res. 18:1742-1746 (2001)); insulin (see Pillion D J et al., I Endocrinology 135:2386-2391 (1994) and Ahsan F. et al., Eur. J. Pharm. Sci. 20:27-34 (2003)); heparin (see Arnold J J et al., J. Pharm. Sci. 91:1707-1714 (2002)) for administration as a nasal spray or nose drops has provided non-invasive and convenient methods of drug delivery that not only reduce the discomfort and risk of infection associated with injection methods, but also fosters higher levels of patient compliance.

The use of various Intravail® transmucosal absorption enhancing agents has extended beyond intranasal delivery of peptides and proteins to include oral, flash-dissolve buccal, and pediatric rectal suppository applications. (See Maggio E T. et al., Expert Opin Drug Deliv 3:529-539 (2006)). Moreover, oral delivery of mouse [D-Leu-4]-OB3 in the presence of Intravail® does not impact negatively on its biological activity, and results in a significant positive influence on energy balance, glycemic control, and bone formation in genetically obese ob/ob mice. In fact, the effects of orally delivered mouse [D-Leu-4]-OB3 in Intravail® on body weight gain, food intake, and serum glucose levels are comparable to, or surpass, those previously seen with ip administration of this peptide and its related analogs. (See Rozhayskaya-Arena M. et al., Endocrinology 141:2501-2517 (2000); Grasso P. et al., Regulatory Pep. 101:123-129 (2001); Grasso P. et al., Diabetes 48:2204-2209 (1999); Grasso P. et al., Endocrinology 138:1413-1418 (1997); Grasso P. et al., Regulatory Peptides 85:93-100, (1999); and Grasso P. et al., Diabetes 48:2204-2209 (1999)).

The pleiotropic nature of leptin action has been previously confirmed. In addition to its role in feeding behavior and energy balance, leptin has now been implicated as an important regulatory molecule in lipid metabolism, hematopoiesis, sympathetic activation, brain development, angiogenesis, immune function, insulin action, ovarian function, reproduction, and bone growth. (See Shimabukuro M. et al., Proc. Natl. Acad. Sci. 94:4637-4641 (1997); Gainsford T. et al., Proc. Natl. Acad. Sci. 93:14563-15568 (1996); Colins S. et al., Nature 380:677 (1996); Steppan C M. et al., Biophys. Biochem. Res. Commun. 256:600-602 (1999); Sierra-Honigmann M R. et al., Science 281:1683-1686 (1998); Lord G M. et al., Nature 394:897-901 (1996); Cohen B. et al., Science 274:1185-1188 (1996); Barash I A. et al., Endocrinology 137:3144-3177 (1996); Considine R V. et al., Curr. Opin. Endocrinol. Diabetes 6:163-169 (1999); Steppan C M. et al., Regulatory Peptides 92:73-78 (2000); Holloway A R. et al., J. Bone Miner. Res. 17:200-209 (2002); and Stravropoulou A. et al., Clin. Chem. Lab. Med. 43:1359-1365 (2005) each of which is incorporated herein by reference).

Using synthetic peptides, and utilizing in vitro and in vivo approaches, peripheral and intracerebroventricular delivery systems, and different animal models, have provided convincing evidence that the entire leptin molecule is not required for its biological activity, and that many of the actions of leptin are more than likely regulated by a domain between amino residues 116 and 130. (See Gonzalez L C. et al., Neuroendocrinology 70:213-220 (1999); Malendowicaz L K. et al., Med. Sci. Res. 27:675-676 (1999); Tena-Sempere M. et al., Eur. J. Endocrinol. 142:406-410 (2000); Malendowicz, L K. et al., Endocr. Res. 26:102-118 (2000); Malendowicz L K. et al., Int. J. Mol. Med. 14:873-877 (2004); Oliveira Jr V X. et al., Regulatory Peptides 127:123-132 (2005); Oliveira Jr V X. et al., J. Pept. Sci. 4:617-25 (2008); and Martins M N C. et al., Regulatory Pept. 153:71-82 (2009)).

In addition to its effects on energy balance and glycemic control, orally delivered mouse [D-Leu-4]-OB3 influences bone formation as well. Plasma or serum levels of osteocalcin, a calcium binding protein synthesized by mature osteoblasts, are used as sensitive and specific markers of osteoblastic activity and bone formation. (See Calvo M S. et al., Endocr. Rev. 17:333-368 (1996)). Those skilled in the art will recognize that bone formation is reduced in cases of malnutrition, starvation, and anorexia nervosa leading to osteoporosis resulting from low bone turnover. (See Himes J H. World Rev. Nutr. Diet. 28:143-187 (1978); Fonseca V A. et al., J. Clin. Pathol. 41:195-197 (1988); and Kawashima, H. et al., Res. Commun. Chem. Pathol. Pharmacol. 33:155-161 (1981)). Acute fasting and chronic food restriction decrease circulating osteocalcin levels. (See Ndiaye B. et al., J. Nutr. 125:1283-1290 (1995); Ndiaye B. et al., Nutr. Res. 13; 71-76 (1993); and Fonseca V A. et al., J. Clin. Pathol. 41:195-197 (1988)).

A number of studies have shown that ob/ob and db/db mice, and Zucker rats display reduced bone mass, mineralization, and bone formation rate when compared to nonobese wild type mice of the same age and sex. (See Foldes J. et al., Int. J. Obes. Relat. Metab. Disord. 16:95-102 (1992) and Goldstone A P. et al., Biochem. Biophys. Res. Commun 295:475-481 (2002)). Leptin has been shown to prevent this bone loss. (See Considine R V. et al., Curr. Opin. Endocrinol. Diabetes 6:163-169 (1999) and Goldstone A P. et al., Leptin prevents the fall in plasma osteocalcin during starvation in male mice. See Biochem. Biophys. Res. Commun 295:475-481 (2002)). As shown herein, serum osteocalcin levels in ob/ob mice were lower than those seen in their sex- and age-matched nonobese counterparts. Further, mouse [D-Leu-4]-OB3 significantly elevated serum osteocalcin to levels higher than those seen in wild type control mice.

These results are similar to those Seen with leptin administered ip in this mouse model (see Goldstone A P. et al., Biochem. Biophys. Res. Commun 295:475-481 (2002)), and indicate that oral delivery of mouse [D-leu-4]-OB3 is as effective as ip leptin administration in preventing bone loss.

In order to assess the effects of orally delivered mouse [D-Leu-4]-OB3 on bone formation in an animal model of malnutrition, calorie intake in both wild type and ob/ob mice was restricted by 40% of normal for 10 days. As expected, this action resulted in significant weight loss, reduced serum glucose, and lower serum osteocalcin levels in both models. Treatment with orally delivered mouse [D-Leu-4]-OB3 significantly elevated serum osteocalcin levels in both calorie restricted wild type and ob/ob mice to levels seen in their counterparts allowed food and water ad libitum. These results are similar to those previously seen in a calorie restricted mouse model treated with ip leptin for five days. (See Goldstone A P. et al., Biochem. Biophys. Res. Commun 295:475-481 (2002)).

This provides in vivo evidence supporting a new physiological role for mouse [D-Leu-4]-OB3 in the regulation of osteoblast activity and bone formation. Worthy of note is the ability of mouse [D-Leu-4]-OB3 to elevate serum osteocalcin in animal models of obesity and malnutrition following oral delivery. Thus, reformulation of mouse [D-Leu-4]-OB3 with Intravail® (or any other suitable delivery system or tansmucosal absorption enhancer known to those skilled in the art) in an oral application has potential not only as an alternative therapy for the treatment of human obesity and some of its associated metabolic dysfunctions, but may also help to prevent some of the bone loss associated with anorexia nervosa and other wasting diseases.

Also described herein are the results of intranasal administration of mouse [D-Leu-4]-OB3 reconstituted in Intravail® to male Swiss Webster mice, which resulted in significantly higher bioavailability than other commonly used injection delivery methods. Specifically, the absorption profile associated with intranasal delivery of mouse [D-Leu-4]-OB3 showed an early peak representing uptake across the nasal mucosa, and a later peak suggesting a gastrointestinal site of absorption. The pharmacodynamic effects of orally administered (by gavage) mouse [D-Leu-4]-OB3 on energy balance, glycemic control, and serum osteocalcin levels in male C57BL/6J wild type and ob/ob mice allowed food and water ad libitum or calorie restricted by 40% of normal intake were also examined.

In wild type mice fed ad libitum, oral delivery of mouse [D-Leu-4]-OB3 reduced body weight gain, food intake, and serum glucose, by 4.4%, 6.8% and 28.2%, respectively. Serum osteocalcin levels and water intake were essentially the same in control and mouse [D-Leu-4]-OB3 treated wild type mice. In ob/ob mice fed ad libitum, mouse [D-Leu-4]-OB3 reduced body weight gain, food intake, water intake, and serum glucose by 11.6%, 16.5%, 22.4% and 24.4%, respectively. Serum osteocalcin levels in ob/ob mice treated with mouse [D-Leu-4]-OB3 were elevated by 161.0% over control levels. Calorie restriction alone caused significant weight loss in both wild type (9.0%) and ob/ob (4.8%) mice.

Treatment with mouse [D-Leu-4]-OB3 did not enhance this weight loss in either wild type or ob/ob mice. Serum glucose levels in wild type and ob/ob mice were significantly reduced by calorie restriction alone. Mouse [D-Leu-4]-OB3 further reduced serum glucose in wild type mice, and normalized levels in ob/ob mice. Calorie restriction alone significantly reduced serum osteocalcin levels by 44.2% in wild type mice, and by 19.1% in ob/ob mice. Mouse [D-Leu-4]-OB3 prevented this decrease in both wild type and ob/ob mice. These results suggest that oral delivery of bioactive mouse [D-Leu-4]-OB3 in Intravail® is possible, and may have potential as an alternative therapy in the treatment of human obesity and some of its associated metabolic dysfunctions, and in the prevention of at least some of the bone loss associated with osteoporosis, anorexia nervosa, and other wasting diseases.

It has also been shown that intranasal administration of mouse [D-Leu-4]-OB3 reconstituted in Intravail® to male Swiss Webster mice resulted in significantly higher bioavailability than other commonly used injection delivery methods. Again, the absorption profile associated with intranasal delivery of mouse [D-Leu-4]-OB3 showed an early peak representing uptake across the nasal mucosa, and a later peak suggesting a gastrointestinal site of absorption.

The pharmacodynamic effects of orally administered (by gavage) mouse [D-Leu-4]-OB3 on energy balance, glycemic control, and serum osteocalcin levels in male C57BL/6J wild type and ob/ob mice allowed food and water ad libitum or calorie restricted by 40% of normal intake have been studied. In wild type mice fed ad libitum, oral delivery of mouse [D-Leu-4]-OB3 reduced body weight gain, food intake, and serum glucose, by 4.4%, 6.8% and 28.2%, respectively. Serum osteocalcin levels and water intake were essentially the same in control and mouse [D-Leu-4]-OB3 treated wild type mice. In ob/ob mice fed ad libitum, mouse [D-Leu-4]-OB3 reduced body weight gain, food intake, water intake, and serum glucose by 11.6%, 16.5%, 22.4% and 24.4%, respectively. Serum osteocalcin levels in ob/ob mice treated with mouse [D-Leu-4]-OB3 were elevated by 62.0% over control levels. Calorie restriction alone caused significant weight loss in both wild type (9.0%) and ob/ob (4.8%) mice. Treatment with mouse [D-Leu-4]-OB3 did not enhance this weight loss in either wild type or ob/ob mice.

Serum glucose levels in wild type and ob/ob mice were significantly reduced by calorie restriction alone. Mouse [D-Leu-4]-OB3 further reduced serum glucose in wild type mice, and normalized levels in ob/ob mice. Calorie restriction alone significantly reduced serum osteocalcin levels by 44.2% in wild type mice, and by 19.1% in ob/ob mice. Mouse [D-Leu-4]-OB3 prevented this decrease in both wild type and ob/ob mice. These results suggest that oral delivery of bioactive mouse [D-Leu-4]-OB3 in Intravail® is possible, and may have potential not only as an alternative therapy in the treatment of human obesity and some of its associated metabolic dysfunctions, but also may help to prevent at least some of the bone loss associated with osteoporosis, anorexia nervosa, and other wasting diseases.

### Recombinant Technologies for Obtaining the Aforementioned Peptides

The aforementioned peptides may be obtained by methods well-known in the art for peptide purification and recombinant peptide expression. For recombinant expression of one or more of the peptides, the nucleic acid containing all or a portion of the nucleotide sequence encoding the peptide may be inserted into an appropriate expression vector (i.e., a vector that contains the necessary elements for the transcription and translation of the inserted peptide coding sequence). In a preferred embodiment, the regulatory elements are heterologous (i.e., not the native gene promoter). Alternately, the necessary transcriptional and translational signals may also be supplied by the native promoter for the genes and/or their flanking regions.

### Host-Vector Systems

A variety of host-vector systems may be utilized to express the peptide coding sequence(s). These include, but are not limited to: (i) mammalian cell systems that are infected with vaccinia virus, adenovirus, and the like; (ii) insect cell systems infected with baculovirus and the like; (iii) yeast containing yeast vectors or (iv) bacteria transformed with bacteriophage, DNA, plasmid DNA, or cosmid DNA. Depending upon the host-vector system utilized, any one of a number of suitable transcription and translation elements may be used.

Any of the methodologies known within the relevant prior art regarding the insertion of nucleic acid fragments into a vector may be utilized to construct expression vectors that contain a chimeric gene comprised of the appropriate transcriptional/translational control signals and peptide-coding sequences. Promoter/enhancer sequences within expression vectors may utilize plant, animal, insect, or fungus regulatory sequences, as provided in the invention.

Promoter/enhancer elements from yeast and other fungi (e.g., the Ga14 promoter, the alcohol dehydrogenase promoter, the phosphoglycerol kinase promoter, the alkaline phosphatase promoter), as well as from animal transcriptional control regions, for example, those that possess tissue specificity and have been used in transgenic animals, may be utilized in the production of peptides of the present invention. Transcriptional control sequences derived from animals include, but are not limited to: (i) the insulin gene control region active within pancreatic β-cells (See, e.g., Hanahan, et al., 1985. Nature 315: 115-122); (ii) the immunoglobulin gene control region active within lymphoid cells (See, e.g., Grosschedl, et al., 1984. Cell 38: 647-658); (iii) the albumin gene control region active within liver (See, e.g., Pinckert, et al., 1987. Genes and Dev 1: 268-276; (iv) the myelin basic protein gene control region active within brain oligodendrocyte cells (See, e.g., Readhead, et al., 1987. Cell 48: 703-712); and (v) the gonadotrophin-releasing hormone gene control region active within the hypothalamus (See, e.g., Mason, et al., 1986. Science 234: 1372-1378), and the like. In a preferred embodiment, a vector is utilized that is comprised of a promoter operably-linked to nucleic acid sequences encoding the aforementioned peptides, one or more origins of replication, and, optionally, one or more selectable markers.

Once the recombinant molecules have been identified and the complex or individual proteins isolated, and a suitable host system and growth conditions have been established, using methods and systems well known within the art, the recombinant expression vectors may be propagated and amplified in-quantity. As previously discussed, expression vectors or their derivatives that can be used include, but are not limited to, human or animal viruses (e.g., vaccinia virus or adenovirus); insect viruses (e.g., baculovirus); yeast vectors; bacteriophage vectors (e.g., lambda phage); plasmid vectors and cosmid vectors.

### Modification

A host cell strain may be selected that modulates the expression of inserted sequences of interest, or modifies or processes expressed peptides encoded by said sequences in the specific manner desired. In addition, expression from certain promoters may be enhanced in the presence of certain inducers in a selected host strain; thus facilitating control of the expression of a genetically-engineered peptides. Moreover, different host cells possess characteristic and specific mechanisms for the translational and post-translational processing and modification (e.g., glycosylation, phosphorylation, and the like) of expressed peptides. Appropriate cell lines or host systems may thus be chosen to ensure the desired modification and processing of the foreign peptide is achieved. For example, peptide expression within a bacterial system can be used to produce an unglycosylated core peptide; whereas expression within mammalian cells ensures “native” glycosylation of a heterologous peptide.

In a specific embodiment of the present invention, the nucleic acids encoding peptides, and peptides consisting of or comprising a fragment of the aforementioned leptin-related sequences that consists of a minimum of 6 contiguous amino acid residues of the aforementioned peptides, are provided herein. Derivatives or analogs of the aforementioned peptides include, but are not limited to, molecules comprising regions that are substantially homologous to the aforementioned peptides in various embodiments, of at least 30%, 40%, 50%, 60%, 70%, 80%, 90% or preferably 95% amino acid identity when: (i) compared to an amino acid sequence of identical size; (ii) compared to an aligned sequence in that the alignment is done by a computer homology program known within the art or (iii) the encoding nucleic acid is capable of hybridizing to a sequence encoding the aforementioned peptides under stringent (preferred), moderately stringent, or non-stringent conditions (see, e.g., supra).

Derivatives of the aforementioned peptides may be produced by alteration of their sequences by substitutions, additions or deletions that result in functionally-equivalent molecules. In a specific embodiment of the present invention, the degeneracy of nucleotide coding sequences allows for the use of other DNA sequences that encode substantially the same amino acid sequence. In another specific embodiment, one or more amino acid residues within the sequence of interest may be substituted by another amino acid of a similar polarity and net charge, thus resulting in a silent alteration. Substitutes for an amino acid within the sequence may be selected from other members of the class to which the amino acid belongs. For example, nonpolar (hydrophobic) amino acids include alanine, leucine, isoleucine, valine, proline, phenylalanine, tryptophan and methionine. Polar neutral amino acids include glycine, serine, threonine, cysteine, tyrosine, asparagine, and glutamine. Positively charged (basic) amino acids include arginine, lysine and histidine. Negatively charged (acidic) amino acids include aspartic acid and glutamic acid.

### Production of Derivatives and Analogs

Derivatives and analogs of the aforementioned peptides of the present invention may be produced by various methodologies known within the art. For example, the polypeptide sequences may be modified by any of numerous methods known within the art. See e.g., Sambrook, et al., 1990. Molecular Cloning: A Laboratory Manual, 2nd ed., (Cold Spring Harbor Laboratory Press; Cold Spring Harbor, N.Y.).

### Isolation and Analysis of the Gene Product or Complex

Once a recombinant cell expressing an aforementioned peptide, or a fragment, homolog, analog or derivative thereof, is identified, the individual gene product or complex may be isolated and analyzed. This is achieved by assays that are based upon the physical and/or functional properties of the peptide or complex, including, but not limited to, radioactive labeling of the product followed by analysis by gel electrophoresis, immunoassay, cross-linking to marker-labeled products, and the like. An aforementioned peptide may be isolated and purified by standard methods known in the art (either from synthetic sources, natural sources or recombinant host cells expressing the peptide/peptide complex) including, but not limited to, column chromatography (e.g., ion exchange, affinity, gel exclusion, reverse-phase, high pressure, fast protein liquid, etc), differential centrifugation, differential solubility, or similar methodologies used for the purification of peptides. Alternatively, once an aforementioned peptide or its derivative is identified, the amino acid sequence of the peptide can be deduced from the nucleic acid sequence of the gene from which it was encoded. Hence, the peptide or its derivative can be synthesized by standard chemical methodologies known in the art. (See, e.g., Hunkapiller, et al., 1984. Nature 310: 105-111).

In a specific embodiment, an aforementioned peptide (whether produced by recombinant DNA techniques, chemical synthesis methods, or by purification from native sources) is made up from peptides, or fragments, analogs or derivatives thereof, that, as their primary amino acid, contain sequences substantially as described herein, as well as peptides substantially homologous thereto.

### Manipulations of the Sequences

Manipulations of the sequences included within the scope of the invention may be made at the peptide level. Included within the scope of the present invention is an aforementioned peptide, or fragments, derivatives, or analogs, that is differentially modified during or after translation or synthesis (e.g., by glycosylation, acetylation, phosphorylation, amidation, derivatization by known protecting/blocking groups, proteolytic cleavage, linkage to an antibody molecule or other cellular ligand, and the like). Any of the numerous chemical modification methodologies known within the art may be utilized including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc. In a specific embodiment, sequences of an aforementioned peptide are modified to include a fluorescent label. In another specific embodiment, an aforementioned peptide is modified by the incorporation of a heterofunctional reagent, wherein such heterofunctional reagent may be used to cross-link the members of the complex.

Production of Peptides—Expression from Tissue Culture Cells

In one embodiment, the invention provides methods of producing any one of the polypeptides set forth in herein, by culturing a cell that contains any one nucleic acid sequence encoding any one of the polypeptides set forth herein under conditions permitting the production of the polypeptide, and recovering the polypeptide from the culture medium or cell culture. Any method known in the art is contemplated for steps needed for production of the peptides including, but not limited to: culturing a cell of choice in an appropriate media; introducing a nucleic acid encoding a peptide of the invention; expressing the peptide from the nucleic acid; secreting the peptide into the culture medium, recovering the peptide from the cell or the culture medium, and purifying the peptide. (See, e.g., Ausubel et al., (Eds). In: CURRENT PROTOCOLS IN MOLECULAR BIOLOGY. J. Wiley and Sons, New York, N.Y. 1998).

The methods of producing any one or more peptide utilized by the methods taught herein involve methods comprising the SEQ ID NOS. identified herein, by introducing a polynucleotide, which encodes, upon expression, for any peptide described herein, into a cell or introducing a peptide coding sequence by homologous recombination into a cell, such that the endogenous regulatory sequence regulates expression of a recombinant peptide gene, to make a peptide production cell and culturing the peptide production cell under culture conditions which result in expression of the peptide. (See, e.g., Ausubel et al., (Eds). In: CURRENT PROTOCOLS IN MOLECULAR BIOLOGY. J. Wiley and Sons, New York, N.Y. 1998).

Cells so treated may then be introduced in vivo for therapeutic purposes by any method known in the art, including, but not limited to, implantation or transplantation of cells into a host subject, wherein the cells may be “naked” or encapsulated prior to implantation. Cells may be screened prior to implantation for various characteristics including, but not limited to, the level of peptide secreted, stability of expression, and the like.

Transgenic animals containing nucleic acids that encode any one or more of the peptides described herein may also be used to express peptides of the invention.

### Chemical Synthesis

Complexes of analogs and derivatives of an aforementioned peptide can be chemically synthesized. For example, a peptide corresponding to a portion of an aforementioned peptide that comprises the desired domain or that mediates the desired activity in vitro, may be synthesized by use of a peptide synthesizer. In cases where natural products are suspected of being mutant or are isolated from new species, the amino acid sequence of an aforementioned protein isolated from the natural source, as well as those expressed in vitro, or from synthesized expression vectors in vivo or in vitro, may be determined from analysis of the DNA sequence, or alternatively, by direct sequencing of the isolated protein. An aforementioned peptide may also be analyzed by hydrophilicity analysis (See, e.g., Hopp and Woods, Proc. Natl. Acad. Sci. USA 78:3824-3828 (1981)) that can be utilized to identify the hydrophobic and hydrophilic regions of the peptides, thus aiding in the design of substrates for experimental manipulation, such as in binding experiments, antibody synthesis, etc.

Secondary structural analysis may also be performed to identify regions of an aforementioned peptide that assume specific structural motifs. (See e.g., Chou and Fasman, Biochem. 13:222-223 (1974). Manipulation, translation, secondary structure prediction, hydrophilicity and hydrophobicity profiles, open reading frame prediction and plotting, and determination of sequence homologies, can be accomplished using computer software programs available in the art. Other methods of structural analysis including, but not limited to, X-ray crystallography (see, e.g., Engstrom, Biochem. Exp. Biol. 11:7-13 (1974)); mass spectroscopy and gas chromatography (see, e.g., METHODS IN PROTEIN SCIENCE, 1997. J. Wiley and Sons, New York, N.Y.) and computer modeling (see, e.g., Fletterick and Zoller, eds. Computer Graphics and Molecular Modeling, In: CURRENT COMMUNICATIONS IN MOLECULAR BIOLOGY, Cold Spring Harbor Laboratory Press, Cold Spring Harbor, N.Y. 1986) may also be employed.

### Methodologies for Screening

The present invention provides methodologies for screening an aforementioned peptide, as well as derivatives, fragments and analogs thereof, for the ability to alter and/or modulate cellular functions, particularly those functions in which an aforementioned peptide have been implicated. These functions include, but are not limited to, weight control; regulation of metabolism; control of signal transduction; and pathological processes, as well as various other biological activities (e.g., binding to antibody against an aforementioned peptide, and the like). The derivatives, fragments or analogs that possess the desired immunogenicity and/or antigenicity may be utilized in immunoassays, for immunization, for inhibition of the activity of an aforementioned peptide, etc. For example, derivatives, fragments or analogs that retain, or alternatively lack or inhibit, a given property of interest may be utilized as inducers, or inhibitors, respectively, of such a property and its physiological correlates. Derivatives, fragments and analogs of an aforementioned peptide may be analyzed for the desired activity or activities by procedures known within the art.

### Production of Antibodies

As disclosed by the present invention herein, the aforementioned peptides, or derivatives, fragments, analogs or homologs thereof, may be utilized as immunogens in the generation of antibodies that immunospecifically bind these peptide components. Such antibodies include, but are not limited to, polyclonal, monoclonal, chimeric, single chain, F_{ab} fragments and an F_{ab} expression library. In a specific embodiment, antibodies to human peptides are disclosed. In another specific embodiment, fragments of the aforementioned peptides are used as immunogens for antibody production. Various procedures known within the art may be used for the production of polyclonal or monoclonal antibodies to an aforementioned peptide, or derivative, fragment, analog or homolog thereof.

For the production of polyclonal antibodies, various host animals may be immunized by injection with the native peptide, or a synthetic variant thereof, or a derivative of the foregoing. Various adjuvants may be used to increase the immunological response and include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.) and human adjuvants such as Bacille Calmette-Guerin and Corynebacterium parvum.

For preparation of monoclonal antibodies directed towards an aforementioned peptide, or derivatives, fragments, analogs or homologs thereof, any technique that provides for the production of antibody molecules by continuous cell line culture may be utilized. Such techniques include, but are not limited to, the hybridoma technique (See Kohler and Milstein, 1975. Nature 256: 495-497); the trioma technique; the human B-cell hybridoma technique (See Kozbor, et al., 1983. Immunol Today 4: 72) and the EBV hybridoma technique to produce human monoclonal antibodies (See Cole, et al., 1985. In: Monoclonal Antibodies and Cancer Therapy, Alan R. Liss, Inc., pp. 77-96). Human monoclonal antibodies may be utilized in the practice of the present invention and may be produced by the use of human hybridomas (See Cote, et al., 1983. Proc Natl Acad Sci USA 80: 2026-2030) or by transforming human B-cells with Epstein Barr Virus in vitro (See Cole, et al., 1985. In: Monoclonal Antibodies and Cancer Therapy (Alan R. Liss, Inc., pp. 77-96).

According to the invention, techniques can be adapted for the production of single-chain antibodies specific to an aforementioned peptide (see, e.g., U.S. Pat. No. 4,946,778). In addition, methodologies can be adapted for the construction of F_{ab} expression libraries (see, e.g., Huse, et al., 1989. Science 246: 1275-1281) to allow rapid and effective identification of monoclonal F_{ab} fragments with the desired specificity for an aforementioned peptide or derivatives, fragments, analogs or homologs thereof. Non-human antibodies can be “humanized” by techniques well known in the art. See e.g., U.S. Pat. No. 5,225,539. Antibody fragments that contain the idiotypes to an aforementioned peptide may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)}₂ fragment; (iii) an F_{ab} fragment generated by the treatment of the antibody molecule with papain and a reducing agent and (iv) Fᵥ fragments.

In one embodiment, methodologies for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an aforementioned peptide is facilitated by generation of hybridomas that bind to the fragment of an aforementioned peptide possessing such a domain. Antibodies that are specific for a domain within an aforementioned peptide, or derivative, fragments, analogs or homologs thereof, are also provided herein.

It should be noted that the aforementioned antibodies may be used in methods known within the art relating to the localization and/or quantitation of an aforementioned peptide (e.g., for use in measuring levels of the peptide within appropriate physiological samples, for use in diagnostic methods, for use in imaging the peptide, and the like). In a given embodiment, antibodies for the aforementioned peptides, or derivatives, fragments, analogs or homologs thereof that contain the antibody derived binding domain, are utilized as pharmacologically compounds (hereinafter “Therapeutics”).

### Immunoassays

The molecules may be utilized in assays (e.g., immunoassays) to detect, prognose, diagnose, or monitor various conditions, diseases, and disorders characterized by aberrant levels of an aforementioned peptide, or monitor the treatment thereof. An “aberrant level” means an increased or decreased level in a sample relative to that present in an analogous sample from an unaffected part of the body, or from a subject not having the disorder. The aforementioned immunoassay may be performed by a methodology comprising contacting a sample derived from a patient with an antibody under conditions such that immunospecific-binding may occur, and subsequently detecting or measuring the amount of any immunospecific-binding by the antibody. In a specific embodiment, an antibody specific for an aforementioned peptide may be used to analyze a tissue or serum sample from a patient for the presence of an aforementioned peptide; wherein an aberrant level of an aforementioned peptide is indicative of a diseased condition. The immunoassays that may be utilized include, but are not limited to, competitive and non-competitive assay systems using techniques such as Western Blots, radioimmunoassays (RIA), enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein-A immunoassays, etc.

### Assays

Methodologies that are well-known within the art (e.g., immunoassays, nucleic acid hybridization assays, biological activity assays, and the like) may be used to determine whether one or more aforementioned peptides are present at either increased or decreased levels, or are absent, within samples derived from patients suffering from a particular disease or disorder, or possessing a predisposition to develop such a disease or disorder, as compared to the levels in samples from subjects not having such disease or disorder or predisposition thereto.

Accordingly, in specific embodiments of the present invention, diseases and disorders that involve increased/decreased levels of activity of one or more leptin or leptin related peptides may be treated with the leptin-related peptides of the present invention, or their ability to respond to said peptides may be screened for, by quantitatively ascertaining increased/decreased levels of: (i) the one or more aforementioned peptides; (ii) the mRNA encoding an aforementioned peptide (iii) the functional activity or (iv) modulation of body weight homeostasis, following administration of the peptides of the present invention.

The present invention additionally provides kits for diagnostic use that are comprised of one or more containers containing an antibody and, optionally, a labeled binding partner to said antibody. The label incorporated into the antibody may include, but is not limited to, a chemiluminescent, enzymatic, fluorescent, colorimetric or radioactive moiety. In another specific embodiment, kits for diagnostic use that are comprised of one or more containers containing modified or unmodified nucleic acids that encode, or alternatively, that are the complement to, an aforementioned peptide and, optionally, a labeled binding partner to said nucleic acids, are also provided. In an alternative specific embodiment, the kit may comprise, in one or more containers, a pair of oligonucleotide primers (e.g., each 6-30 nucleotides in length) that are capable of acting as amplification primers for polymerase chain reaction (PCR; See, e.g., Innis, et al., 1990. PCR PROTOCOLS, Academic Press, Inc., San Diego, Calif.), ligase chain reaction, cyclic probe reaction, and the like, or other methods known within the art. The kit may, optionally, further comprise a predetermined amount of a purified aforementioned peptide, or nucleic acids thereof, for use as a diagnostic, standard, or control in the aforementioned assays.

### Gene Therapy

In a specific embodiment of the present invention, nucleic acids comprising a sequence that encodes an aforementioned peptide, or functional derivatives thereof, are administered to modulate homeostasis of body weight and adipose tissue mass by way of gene therapy. In more specific embodiments, a nucleic acid or nucleic acids encoding an aforementioned peptide, or functional derivatives thereof, are administered by way of gene therapy. Gene therapy refers to therapy that is performed by the administration of a specific nucleic acid to a subject. In this embodiment of the present invention, the nucleic acid produces its encoded peptide(s), which then serve to exert a therapeutic effect by modulating function of an aforementioned disease or disorder. Any of the methodologies relating to gene therapy available within the art may be used in the practice of the present invention. (See e.g., Goldspiel, et al., Clin. Pharm. 12: 488-505 (1993)).

The Therapeutic comprises a nucleic acid that is part of an expression vector expressing both of the aforementioned peptides, or fragments, derivatives or analogs thereof, within a suitable host. In a specific embodiment, such a nucleic acid possesses a promoter that is operably-linked to coding region(s) of an aforementioned peptide. Said promoter may be inducible or constitutive, and, optionally, tissue-specific. In another specific embodiment, a nucleic acid molecule is used in which coding sequences (and any other desired sequences) are flanked by regions that promote homologous recombination at a desired site within the genome, thus providing for intra-chromosomal expression of nucleic acids. (See e.g., Koller and Smithies, 1989. Proc Natl Acad Sci USA 86: 8932-8935).

Delivery of the Therapeutic nucleic acid into a patient may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. In a specific embodiment of the present invention, a nucleic acid is directly administered in vivo, where it is expressed to produce the encoded product. This may be accomplished by any of numerous methods known in the art including, but not limited to, constructing said nucleic acid as part of an appropriate nucleic acid expression vector and administering the same in a manner such that it becomes intracellular (e.g., by infection using a defective or attenuated retroviral or other viral vector; see U.S. Pat. No. 4,980,286); directly injecting naked DNA; using microparticle bombardment (e.g., a “Gene Gun®; Biolistic, DuPont); coating said nucleic acids with lipids; using associated cell-surface receptors/transfecting agents; encapsulating in liposomes, microparticles, or microcapsules; administering it in linkage to a peptide that is known to enter the nucleus; or by administering it in linkage to a ligand predisposed to receptor-mediated endocytosis (see, e.g., Wu and Wu, 1987. J Biol Chem 262: 4429-4432), which can be used to “target” cell types that specifically express the receptors of interest, etc.

In another specific embodiment of the present invention, a nucleic acid-ligand complex may be produced in which the ligand comprises a fusogenic viral peptide designed so as to disrupt endosomes, thus allowing the nucleic acid to avoid subsequent lysosomal degradation. In yet another specific embodiment, the nucleic acid may be targeted in vivo for cell-specific endocytosis and expression, by targeting a specific receptor. (See e.g., PCT Publications WO 92/06180; WO93/14188 and WO 93/20221). Alternatively, the nucleic acid may be introduced intracellularly and incorporated within a host cell genome for expression by homologous recombination. (See e.g., Zijlstra, et al., 1989. Nature 342: 435-438).

In another specific embodiment, a viral vector that contains nucleic acids encoding an aforementioned peptide is utilized. For example, retroviral vectors may be employed (see, e.g., Miller, et al., 1993. Meth Enzymol 217: 581-599) that have been modified to delete those retroviral-specific sequences that are not required for packaging of the viral genome, with its subsequent integration into host cell DNA. Nucleic acids may be cloned into a vector that facilitates delivery of the genes into a patient. (See e.g., Boesen, et al., 1994. Biotherapy 6: 291-302; Kiem, et al., 1994. Blood 83: 1467-1473). Additionally, adenovirus may be used as an especially efficacious “vehicle” for the delivery of genes to the respiratory epithelia. Other targets for adenovirus-based delivery systems are liver, central nervous system, endothelial cells, and muscle. Adenoviruses also possess advantageous abilities to infect non-dividing cells. For a review see, e.g., Kozarsky and Wilson, 1993. Curr Opin Gen Develop 3: 499-503. Adenovirus-associated virus (AAV) has also been proposed for use in gene therapy. (See e.g., Walsh, et al., 1993. Proc Soc Exp Biol Med 204: 289-300).

An additional approach to gene therapy in the practice of the present invention involves transferring a gene into cells in in vitro tissue culture by such methods as electroporation, lipofection, calcium phosphate-mediated transfection, viral infection, or the like. Generally, the methodology of transfer includes the concomitant transfer of a selectable marker to the cells. The cells are then placed under selection pressure (e.g., antibiotic resistance) so as to facilitate the isolation of those cells that have taken up, and are expressing, the transferred gene. Those cells are then delivered to a patient. In a specific embodiment, prior to the in vivo administration of the resulting recombinant cell, the nucleic acid is introduced into a cell by any method known within the art including, but not limited to: transfection, electroporation, microinjection, infection with a viral or bacteriophage vector containing the nucleic acid sequences of interest, cell fusion, chromosome-mediated gene transfer, microcell-mediated gene transfer, spheroplast fusion, and similar methodologies that ensure that the necessary developmental and physiological functions of the recipient cells are not disrupted by the transfer. (See e.g., Loeffler and Behr, 1993. Meth Enzymol 217: 599-618). The chosen technique should provide for the stable transfer of the nucleic acid to the cell, such that the nucleic acid is expressible by the cell. Preferably, said transferred nucleic acid is heritable and expressible by the cell progeny.

In preferred embodiments of the present invention, the resulting recombinant cells may be delivered to a patient by various methods known within the art including, but not limited to, injection of epithelial cells (e.g., subcutaneously), application of recombinant skin cells as a skin graft onto the patient, and intravenous injection of recombinant blood cells (e.g., hematopoietic stem or progenitor cells). The total amount of cells that are envisioned for use depend upon the desired effect, patient state, and the like, and may be determined by one skilled within the art.

Cells into which a nucleic acid can be introduced for purposes of gene therapy encompass any desired, available cell type, and may be xenogeneic, heterogeneic, syngeneic, or autogeneic. Cell types include, but are not limited to, differentiated cells such as epithelial cells, endothelial cells, keratinocytes, fibroblasts, muscle cells, hepatocytes and blood cells, or various stem or progenitor cells, in particular embryonic heart muscle cells, liver stem cells (see PCT Patent Publication WO 94/08598), neural stem cells (see Stemple and Anderson, 1992, Cell 71: 973-985), hematopoietic stem or progenitor cells, e.g., as obtained from bone marrow, umbilical cord blood, peripheral blood, fetal liver, and the like. In a preferred embodiment, the cells utilized for gene therapy are autologous to the patient.

In a specific embodiment in which recombinant cells are used in gene therapy, stem or progenitor cells that can be isolated and maintained in vitro may be utilized. Such stem cells include, but are not limited to, hematopoietic stem cells (HSC), stem cells of epithelial tissues, and neural stem cells (See, e.g., Stemple and Anderson, 1992. Cell 71: 973-985). With respect to HSCs, any technique that provides for the isolation, propagation, and maintenance in vitro of HSC may be used in this specific embodiment of the invention. As previously discussed, the HSCs utilized for gene therapy are, preferably but not by way of limitation, autologous to the patient. When used, non-autologous HSCs are, preferably but not by way of limitation, utilized in conjunction with a method of suppressing transplantation immune reactions of the future host/patient. See e.g., Kodo, et al., 1984. Clin Invest 73: 1377-1384. In a preferred embodiment, HSCs may be highly enriched (or produced in a substantially-pure form), by any techniques known within the art, prior to administration to the patient. See e.g., Witlock and Witte, 1982. Proc. Natl. Acad. Sci. USA 79: 3608-3612.

### Pharmaceutical Pack or Kit

The present invention also provides a pharmaceutical pack or kit, comprising one or more containers filled with one or more of the ingredients of the pharmaceutical compositions and Therapeutics of the present invention. Optionally associated with such container(s) may be a notice in the form prescribed by a governmental agency regulating the manufacture, use or sale of pharmaceuticals or biological products, which notice reflects approval by the agency of manufacture, use or sale for human administration.

### Cultured Cells

Cells may be cultured ex vivo in the presence of peptides of the present invention in order to proliferate or to produce a desired effect on or activity in such cells. Treated cells can then be introduced in vivo for therapeutic purposes.

Contemplated within the invention is a method of identifying a modulator and/or potential modulator of body mass homeostasis or serum osteocalcin levels in situ by contacting a cell with the presence or absence of peptide, the peptide comprising any one or more of the peptides described herein; determining the level of effect in cells so contacted compared to cells not so contacted; wherein when an increase or decrease in desired effect is determined in the presence of the peptide relative to in the absence of the peptide, the peptide is identified as a potential modulator of body mass homeostasis or serum osteocalcin levels.

Also contemplated within the invention is a method of identifying a modulator and/or potential modulator of body mass homeostasis or serum osteocalcin levels in vivo by administering to a test animal doses of at least one peptide of the invention and comparing said animal to a placebo control animal over a prescribed time period, wherein the peptide comprises any one or more of the peptides described herein; determining the level of modulation in body homeostasis of the test animal compared to the control during the prescribed time period; wherein when an increase or decrease in desired effect is determined in the presence of the peptide relative to in the absence of the peptide, the peptide is identified as a potential modulator of body mass homeostasis or serum osteocalcin levels. A peptide that causes the test animal to lose weight relative to the control animal may be selected as a drug that is useful in a weight loss diet regimen.

### Determination of the Biological Effect of the Therapeutic

In preferred embodiments of the present invention, suitable in vitro or in vivo assays are utilized to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon said cell type(s). Compounds for use in therapy may be tested in suitable animal model systems including, but not limited to rats, mice, chicken, cows, monkeys, rabbits, and the like, prior to testing in human subjects. In a preferred embodiment, genetically obese C57BL/6J ob/ob or C57BLKS/J-m db/db mice are used. Similarly, for in vivo testing, any of the animal model system known in the art may be used prior to administration to human subjects.

### Pharmaceutical Compositions

A peptide of the present invention (derived from whatever source defined herein, including without limitation from synthetic, recombinant and non-recombinant sources) may be used in a pharmaceutical composition when combined with a pharmaceutically acceptable carrier. Such compositions comprise a therapeutically-effective amount of a Therapeutic, and a pharmaceutically acceptable carrier. Such a composition may also be comprised of (in addition to peptide and a carrier) diluents, fillers, salts, buffers, stabilizers, solubilizers, and other materials well known in the art. The characteristics of the carrier will depend on the route of administration.

A peptide of the present invention may be active in multimers (e.g., heterodimers or homodimers) or complexes with itself or other peptides. As a result, pharmaceutical compositions of the invention may comprise a peptide of the invention in such multimeric or complexed form. More particularly, the pharmaceutical composition may also contain pharmaceutically acceptable carrier such as a drug delivery system. In various embodiments, the drug delivery system is a transmucosal absorption enhancer. For example, the transmucosal absorption enhancer is Intravail®.

### Methods of Administration

Suitable methods of administration include, but are not limited to, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local.

In addition, it may be advantageous to administer the Therapeutic into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant.

### Delivery

Various delivery systems are known and can be used to administer a Therapeutic of the present invention including, but not limited to: (i) encapsulation in liposomes, microparticles, microcapsules; (ii) recombinant cells capable of expressing the Therapeutic; (iii) receptor-mediated endocytosis (see, e.g., Wu and Wu, 1987. J Biol Chem 262:4429-4432); (iv) construction of a Therapeutic nucleic acid as part of a retroviral or other vector, and the like.

In one embodiment of the present invention, the Therapeutic may be delivered in a vesicle, in particular a liposome. In a liposome, the peptide of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028; and U.S. Pat. No. 4,737,323, all of which are incorporated herein by reference.

In yet another embodiment, the Therapeutic can be delivered in a controlled release system including, but not limited to: a delivery pump (see, e.g., Saudek, et al., 1989. New Engl J Med 321:574 and a semi-permeable polymeric material (see, e.g., Howard, et al., 1989. J Neurosurg 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, Fla.).

In a specific embodiment of the present invention, where the Therapeutic is a nucleic acid encoding a peptide, the Therapeutic nucleic acid may be administered in vivo to promote expression of its encoded peptide, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (see, e.g., Joliot, et al., 1991. Proc Natl Acad Sci USA 88:1864-1868), and the like. Alternatively, a nucleic acid Therapeutic can be introduced intracellularly and incorporated within host cell DNA for expression, by homologous recombination.

### Dosage

The amount of the Therapeutic of the invention which will be effective in the treatment of a particular disorder or condition or to achieve a desired effect will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of peptide of the present invention with which to treat each individual patient. Initially, the attending physician will administer low doses of peptide of the present invention and observe the patient's response. Larger doses of peptide of the present invention may be administered until the optimal therapeutic effect is obtained for the patient, and at that point the dosage is not increased further. However, suitable dosage ranges for administration of the Therapeutics of the present invention are generally about 5-500 micrograms (μg) of active compound per kilogram (Kg) body weight. Suitable dosage ranges for intranasal administration are generally about 0.01 pg/kg body weight to 1 mg/kg body weight. Suitable dosage ranges for oral administration are generally 0.01 pg/kg body weight to 1 mg/kg body weight and are generally taken once or twice daily. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

### Duration

The duration of any intravenous therapy using the pharmaceutical composition of the present invention will vary, depending on the severity of the disease being treated and the condition and potential idiosyncratic response of each individual patient. It is contemplated that the duration of each application of the peptide of the present invention will be in the range of 12 to 24 hours of continuous intravenous administration. Ultimately the attending physician will decide on the appropriate duration of intravenous therapy using the pharmaceutical composition of the present invention.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

The invention will be further described in the following examples, which do not limit the scope of the invention described in the claims.

### EXAMPLES

### Example 1

The results of earlier preclinical studies with mouse [D-Leu-4]-OB3 (see U.S. Pat. Nos. 6,777,388; 7,186,694; 7,208,572B2; Australian Patent number 772,278), demonstrate that a synthetic peptide amide with leptin-like activity, when administered via intraperitoneal (ip) delivery significantly improves a number of metabolic dysfunctions associated with the obesity syndrome in the ob/ob mouse model. (See Rozhayskaya-Arena M. et al., Endocrinology 141:2501-2517 (2000) and Grasso P. et al., Regulatory Pep. 101:123-129 (2001)).

More recently, it has been shown that intranasal delivery of mouse [D-Leu-4]-OB3 in Intravail® (Aegis Therapeutics, San Diego, Calif.), a patented transmucosal absorption enhancing agent, results in significantly higher bioavailability of mouse [D-Leu-4]-OB3 when compared to ip and other commonly used injection methods of drug delivery. (See Novakovic Z M. et al., Regulatory Peptides 154:107-111 (2009)).

An unexpected outcome of this study was the appearance of a biphasic absorption profile associated with intranasal delivery of mouse [D-Leu-4]-OB3, which was not observed in the absorption profiles associated with ip, subcutaneous (sc), or intramuscular (im) administration. The time course of this profile suggested a two-compartment model of peptide distribution in which the early peak may represent a very rapid systemic uptake of mouse [D-Leu-4]-OB3 across the nasal mucosa, and the later peak much slower gastrointestinal absorption. Gastrointestinal absorption of mouse [D-Leu-4]-OB3 does occur. The peptide's bioavailability is significantly improved by using Intravail®. In the present study, it has been shown that mouse [D-Leu-4]-OB3 retains bioactivity when given orally by gavage, and exerts its effects on energy balance, glycemic control, and serum osteocalcin levels in wild type and genetically obese C57BL/6J ob/ob mice.

### Materials and Methods

### Animal Procedures

### Housing

Six week-old male C57BL/6J wild type and ob/ob mice were obtained from Jackson Laboratories (Bar Harbor, Me., USA). The animals were housed individually polycarbonate cages fitted with stainless steel wire lids and air filters, and supported on ventilated racks (Thoren Caging Systems, Hazelton, Pa., USA) in the Albany Medical College Animal Resources Facility. The mice were maintained at a constant temperature (24° C.) with lights on from 07:00 to 19:00, and allowed food and water ad libitum for 6 days following arrival. During the test period, mice were fed ad libitum or calorie restricted to 60% of normal. Water intake was allowed ad libitum.

### Feeding and Weighing Schedule

On day 1 of the study, and on each day thereafter, a water bottle containing 200 ml of water was added to each cage between 09:00 and 10:00 h. Mice fed ad libitum were given 200 g of pelleted rodent diet (Prolab Rat, Mouse, Hamster 3000, St. Louis, Mo.; 22% crude protein, 5% crude fat, 5% fiber, 6% ash, 2.5% additional minerals) between 09:00 and 10:00 h each day. The mice were weighed once daily between 09:00 and 10:00 h on an Acculab V-333 electronic balance (Cole-Parmer, Vernon Hills, Ill., USA). Calorie-restricted wild type and ob/ob mice received 60% of normal daily intake. Twenty four hours later, food and water remaining in the cages was measured to the nearest 0.1 g and 0.1 ml, respectively. To assure fasting glucose levels on days in which blood glucose was measured, food was removed from the cages between 09:00 and 10:00 h, and replaced immediately before the beginning of the dark cycle.

### Peptide Administration

Mouse [D-Leu-4]OB3 was prepared commercially as a C-terminal amide by Bachem (Torrance, Calif., USA). The peptide was dissolved in 0.3% Intravail A3® (Aegis Therapeutics, San Diego, Calif. USA) reconstituted in water, and administered by gavage once daily for 10 days at a concentration of 1 mg/200 μl immediately before the start of the dark cycle. Is has previously been shown that this is the optimum concentration of mouse [D-Leu-4]-OB3 and its related bioactive peptide amides, for regulating energy expenditure, glucose levels, and insulin sensitivity in two genetically obese mouse models (see Rozhayskaya-Arena M. et al., Endocrinology 141:2501-2517 (2000); Grasso P. et al. Regulatory Pep. 101:123-129 (2001); Grasso P. et al., Diabetes 48:2204-2209 (1999), Grasso P. et al., Endocrinology 138:1413-1418 (1997); Grasso P. et al., Regulatory Peptides 85:93-100 (1999); and Grasso P. et al., Diabetes 48:2204-2209 (1999)). Vehicle injected control mice received 200 ul of Intravail A3® only.

### Measurement of Blood Glucose

Initial blood samples were drawn by snipping the end of the tail of each mouse at the beginning of the study (day 0); subsequent samples were obtained by gently removing the scab. Blood was taken 1 h before the onset of the dark period at the beginning of the study, and after 2, 4, 6, 8, and 10 days of treatment. The blood was applied to a test strip, and glucose levels were measured with a Glucometer Elite glucose meter (Bayer, Elkhart, Ind., USA).

### Collection of Blood and Serum Preparation

At the end of the study, the mice were anesthetized with isoflurane (5%) and exsanguinated by cardiac puncture. Euthanasia was confirmed by cervical dislocation. The blood was collected in sterile nonheparinized plastic centrifuge tubes and allowed to stand at room temperature for 1 h. The clotted blood was rimmed from the walls of the tubes with sterile wooden applicator sticks. Individual serum samples were prepared by centrifugation for 30 min at 2600×g in an Eppendorf 5702R, A-4-38 rotor (Eppendorf North America, Westbury, N.Y., USA). The serum samples in each experimental group (n=6) were pooled and stored frozen until assayed for osteocalcin content.

All of these animal procedures were approved by the Albany Medical College Animal Care and Use Committee, and were performed in accordance with relevant guidelines and regulations.

### Serum Osteocalcin Measurement

Serum osteocalcin in the pooled samples was assayed in triplicate with a mouse osteocalcin ELISA kit obtained from Biomedical Technologies, Inc. (Stoughton, Mass., USA) according to the instructions supplied by the manufacturer.

### Statistical Analysis

Changes in body weight, food and water intake, and serum glucose and osteocalcin levels were compared by repeated measures analysis of variance (ANOVA) using the statistics program SigmaStat® 3.0 for Windows (SPSS, Inc. Chicago, Ill., USA). Differences were considered significant when P<0.05.

Effects of Oral Delivery of Mouse [D-Leu-4]-OB3 on Body Weight Gain, Food and Water Intake, and Serum Glucose and Osteocalcin Levels in Wild Type and ob/ob Mice Allowed Food and Water Ad Libitum

### Effects of Body Weight Gain

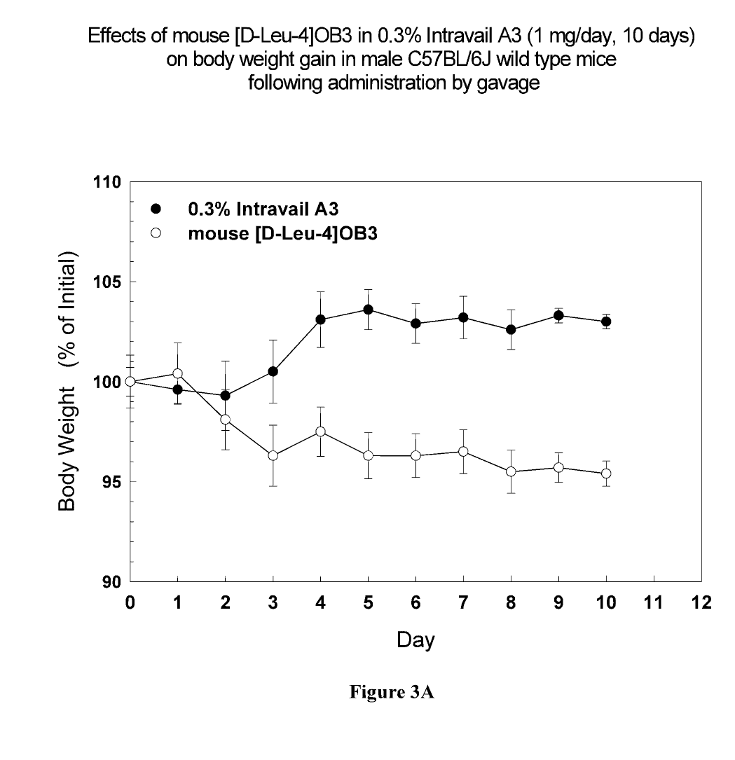

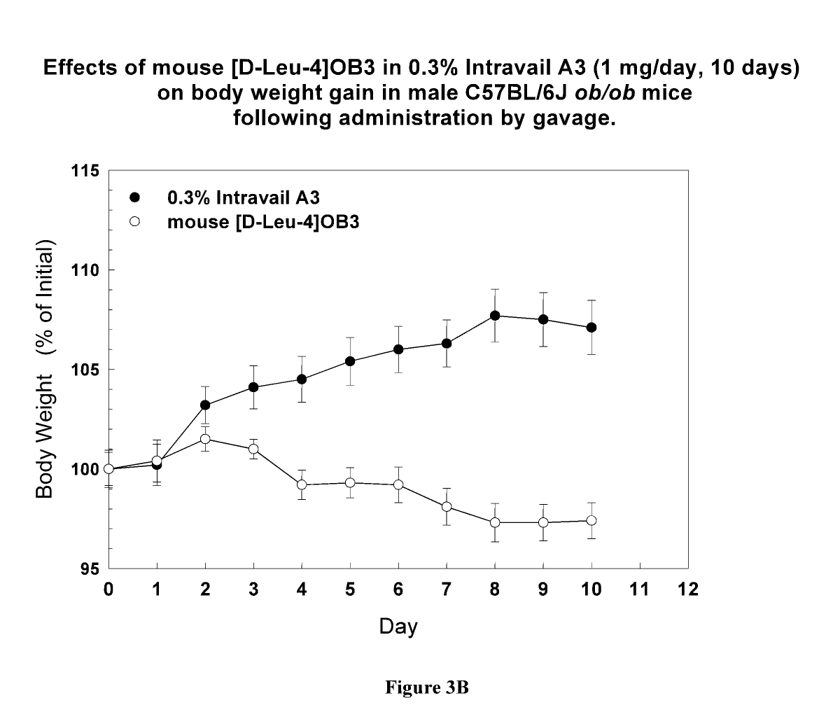

The effects of mouse [D-Leu-4]-OB3 given by gavage to wild type and ob/ob mice allowed food and water ad libitum are shown in FIGS. 3A and 3B, respectively. After 10 days of receiving Intravail® alone, wild type mice were 3.4% heavier than they were at the beginning of the study, while mice treated with mouse [D-Leu-4]-OB3 had lost 4.4% of their initial body weight, and were significantly lighter (P<0.001) than their untreated counterparts (FIG. 3A). The same pattern was Seen in ob/ob mice. Ob/ob mice receiving Intravail® alone for 10 days were 7.9% heavier than they were at the beginning of the study, while ob/ob mice receiving mouse [D-Leu-4]-OB3 lost 3.7% of their initial body weight and were also significantly (P<0.001) lighter than their untreated counterparts (FIG. 3B).

### Effects on Food and Water Intake

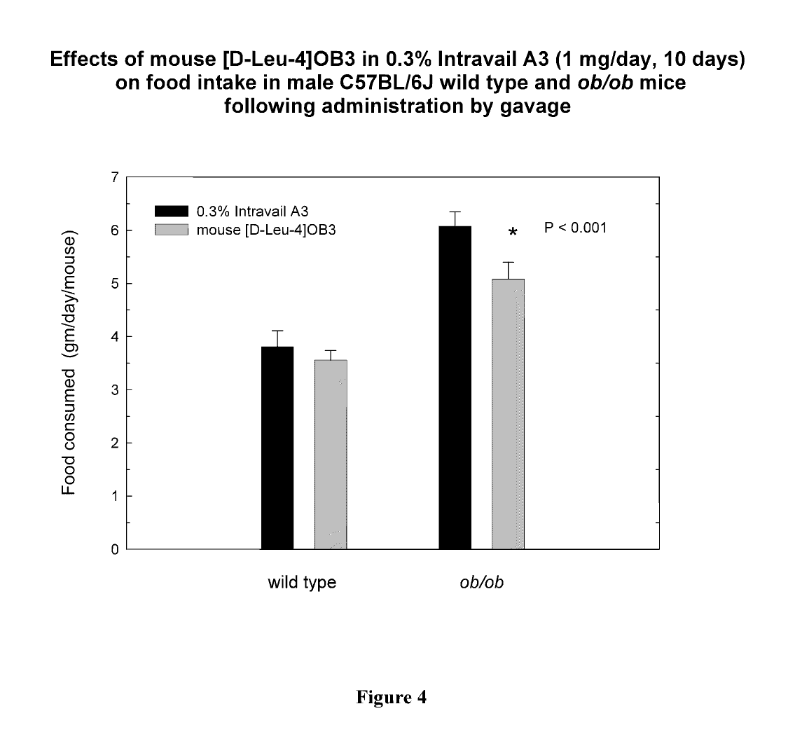

The effects of mouse [D-Leu-4]-OB3 on food intake in wild type and ob/ob mice are shown in FIG. 4. The decrease in body weight Seen in wild type mice receiving mouse [D-Leu-4]-OB3 was not associated with any significant difference in daily food intake when compared to Intravail® treated controls. Daily food intake of ob/ob mice treated with mouse [D-Leu-4]-OB3, however, was significantly (P<0.001) less when compared to ob/ob mice receiving Intravail® alone.

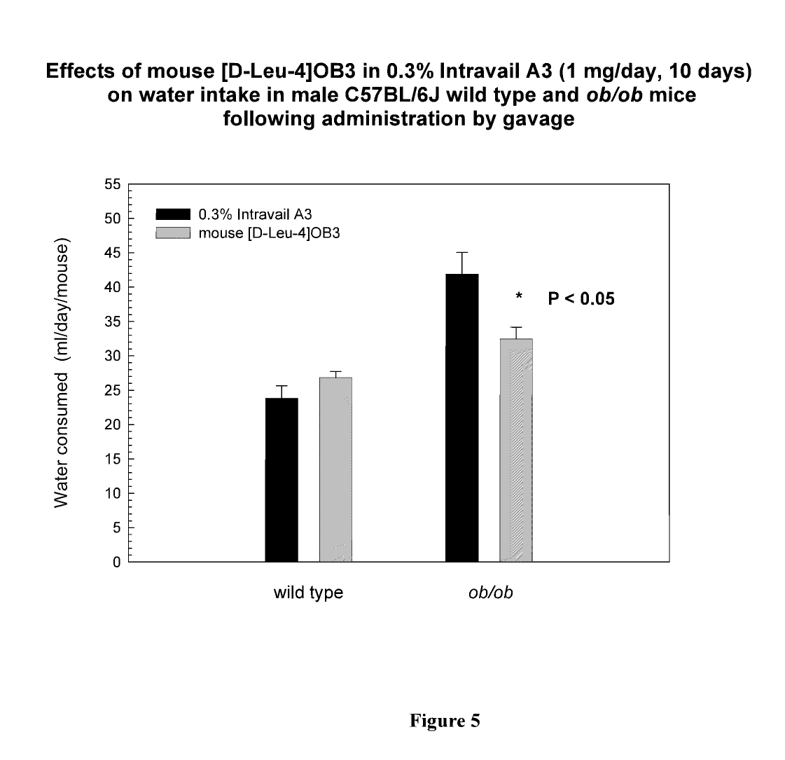

The effects of mouse [D-Leu-4]-OB3 on daily water consumption in wild type and ob/ob mice are shown in FIG. 5. While no significant difference in water intake was observed in wild type mice receiving mouse [D-Leu-4]-OB3 compared to Intravail® treated controls, ob/ob mice receiving mouse [D-Leu-4]-OB3 consumed significantly (P<0.05) less water per day than their Intravail® treated counterparts.

### Effects on Serum Glucose Levels

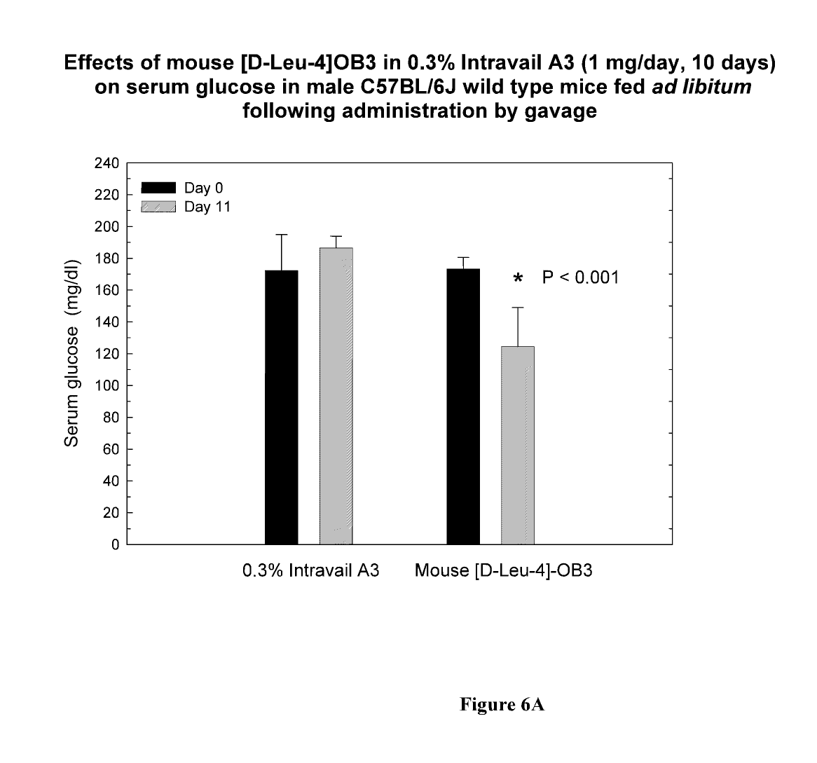

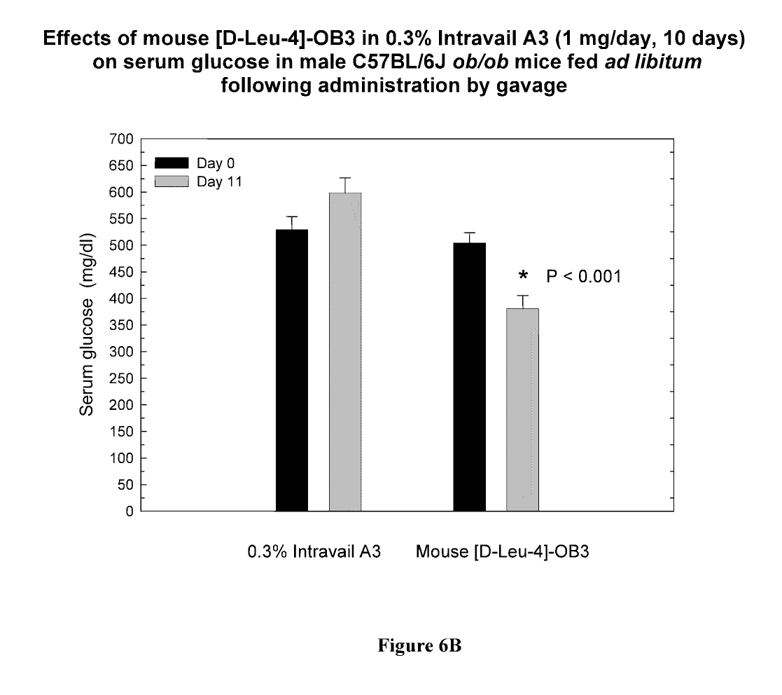

The effects of mouse [D-Leu-4]-OB3 on serum glucose levels in wild type and ob/ob mice are shown in FIGS. 6A and 6B, respectively. In wild type mice (FIG. 6A), serum glucose levels were essentially the same after 10 days of treatment with Intravail® alone. Serum glucose was significantly (P<0.001) reduced by treatment with mouse [D-Leu-4]-OB3 for 10 days.

ob/ob mice (FIG. 6B) treated with Intravail® alone showed higher, but not significant, glucose levels after 10 days of treatment, presumably associated with their increased body weight. Treatment with mouse [D-Leu-4]-OB3 for 10 days significantly (P<0.001) reduced serum glucose levels, but not to normal levels.

### Effects on Serum Osteocalcin Levels

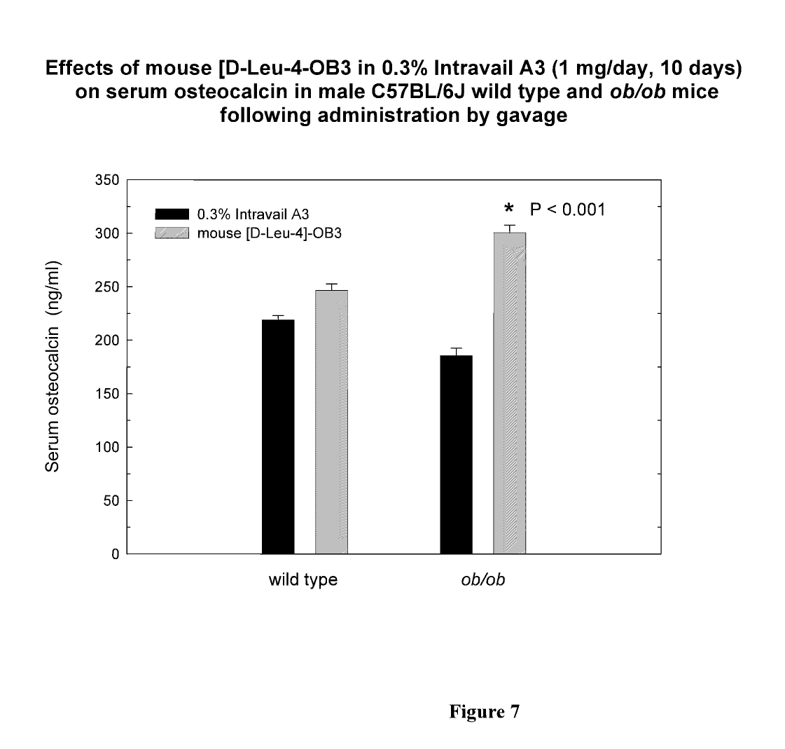

Treatment of wild type mice with mouse [D-Leu-4]-OB3 for 10 days resulted in slightly elevated serum osteocalcin levels compared to Intravail® treated controls. In ob/ob mice with osteocalcin levels approximately 15% lower than their nonobese counterparts, mouse [D-Leu-4]-OB3 significantly (P<0.001) elevated serum osteocalcin by 62% after 10 days of treatment (FIG. 7).

The effects of mouse [D-Leu-4]-OB3 on C57BL/6J wild type and ob/ob mice allowed food and water ad libitum are summarized in Table 1.

TABLE 1
Effects of mouse [D-Leu-4]-OB3 (1 mg/day, 10 days, gavage)
in ad libitum fed male C57BL/6J wild type and ob/ob mice.

| | |
|---|---|
| | Mouse |
| Control | [D-Leu-4]-OB3 |

Wild type

| | | |
|---|---|---|
| Initial body weight (g) | 20.5 ± 0.3 | 22.6 ± 0.8 |
| Final body weight (g) | 21.2 ± 0.3 | 21.6 ± 0.7 |
| Body weight (% of initial) | 103.4 ± 0.7 | 95.6 ± 0.5 |
| Initial serum glucose (mg/dl) | 172.2 ± 22.7 | 173.3 ± 7.3 |
| Final serum glucose (mg/dl) | 186.5 ± 6.8 | 124.5 ± 24.6 |
| Serum osteocalcin (ng/ml) | 218.6 ± 4.2 | 246.6 ± 6.0 |

ob/ob

| | | |
|---|---|---|
| Initial body weight (g) | 29.1 ± 0.9 | 35.5 ± 0.8 |
| Final body weight (g) | 31.4 ± 1.4 | 34.2 ± 0.9 |
| Body weight (% of initial) | 107.9 ± 0.8 | 96.3 ± 0.4 |
| Initial serum glucose (mg/dl) | 529.3 ± 24.5 | 503.9 ± 19.6 |
| Final serum glucose (mg/dl) | 598.6 ± 27.9 | 380.5 ± 24.3 |
| Serum osteocalcin (ng/ml) | 185.5 ± 7.0 | 300.5 ± 7.0 |

Effects of Oral Delivery of Mouse [D-Leu-4]-OB3 on Body Weight Gain, Food and Water Intake, and Serum Glucose and Osteocalcin Levels in Calorie Restricted Wild Type and ob/ob Mice

### Effects of Body Weight Gain

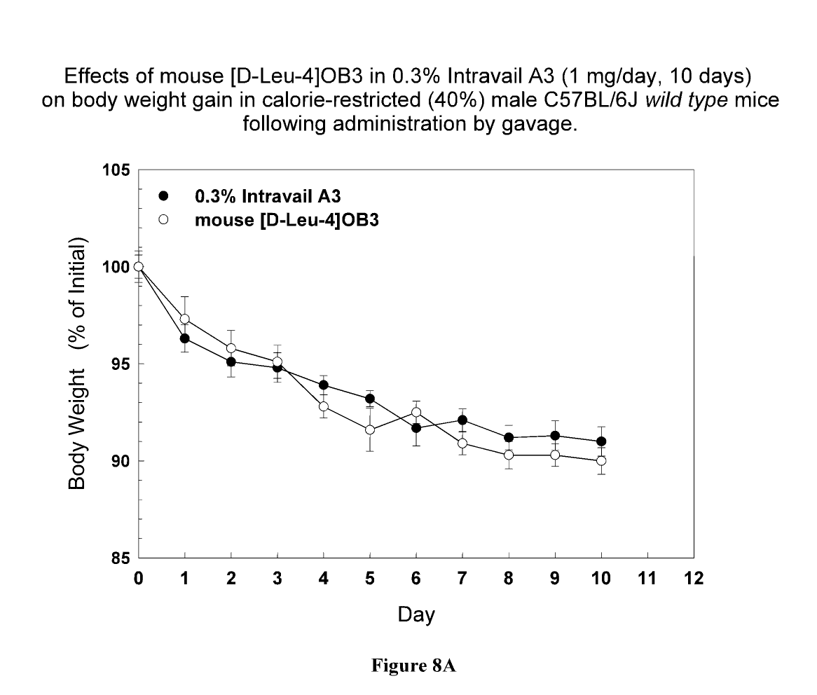

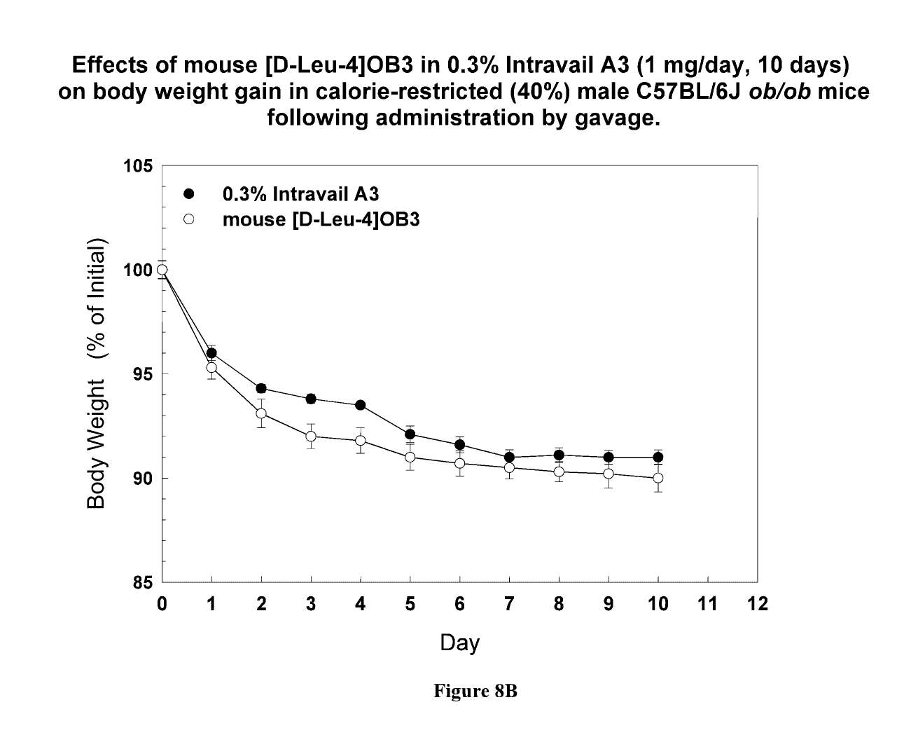

The effects mouse [D-Leu-4]-OB3 on body weight gain in wild type and ob/ob mice in which food intake was restricted to 60% of normal are shown in FIGS. 8A and 8B, respectively. As expected, 10 days of calorie restriction alone resulted in significant (P<0.001) weight loss in both wild type and ob/ob mice when compared to their ad libitum fed counterparts. Weight loss in both Intravail® treated mice and those receiving mouse [D-leu-4]-OB3 was essentially the same throughout the course of the study. (FIG. 8A).

Weight loss was essentially the same in calorie restricted ob/ob mice receiving either Intravail® alone or mouse [D-Leu-4]-OB3 for 10 days. (FIG. 8B).

### Effects on Serum Glucose Levels

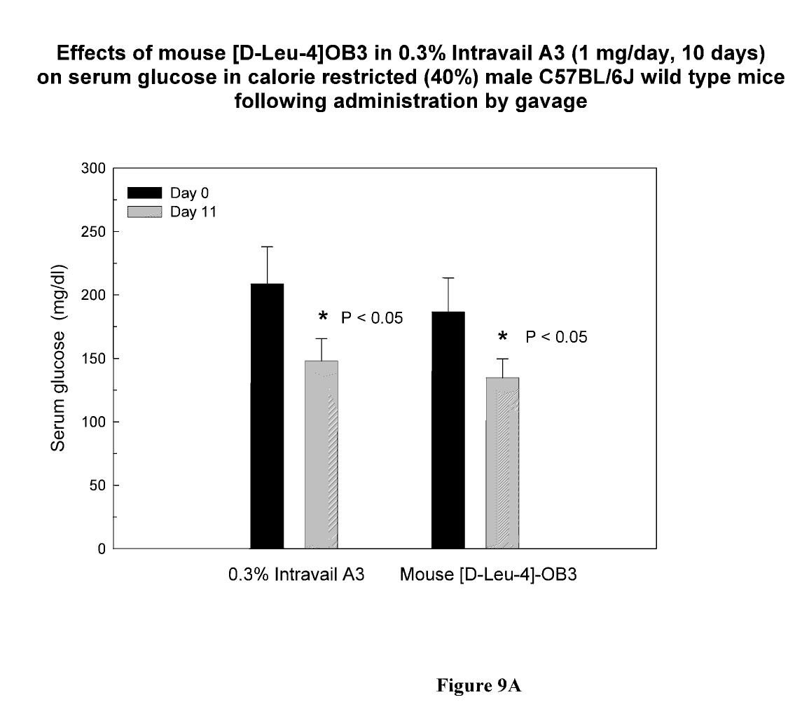

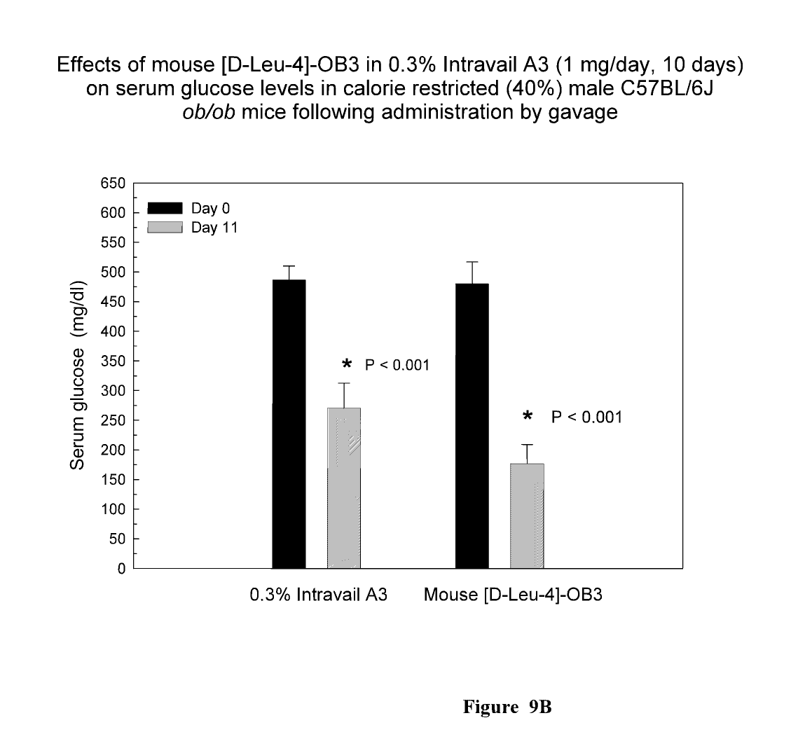

The effects of mouse [D-Leu-4]-OB3 on serum glucose levels in calorie restricted wild type and ob/ob mice are shown in FIGS. 9A and 9B, respectively. In wild type mice, serum glucose levels were significantly (P<0.05) lower after 10 days of treatment with Intravail® alone. Mouse [D-Leu-4]-OB3 did not further reduce serum glucose levels (FIG. 9A).

As expected, calorie restriction significantly (P<0.001) reduced, but did not normalize, serum glucose levels in ob/ob mice treated with Intravail® alone. Treatment with mouse [D-Leu-4]-OB3 for 10 days, however, significantly (P<0.001) reduced serum glucose levels to levels Seen in wild type mice allowed food ad libitum (FIG. 9B).

### Effects on Serum Osteocalcin Levels

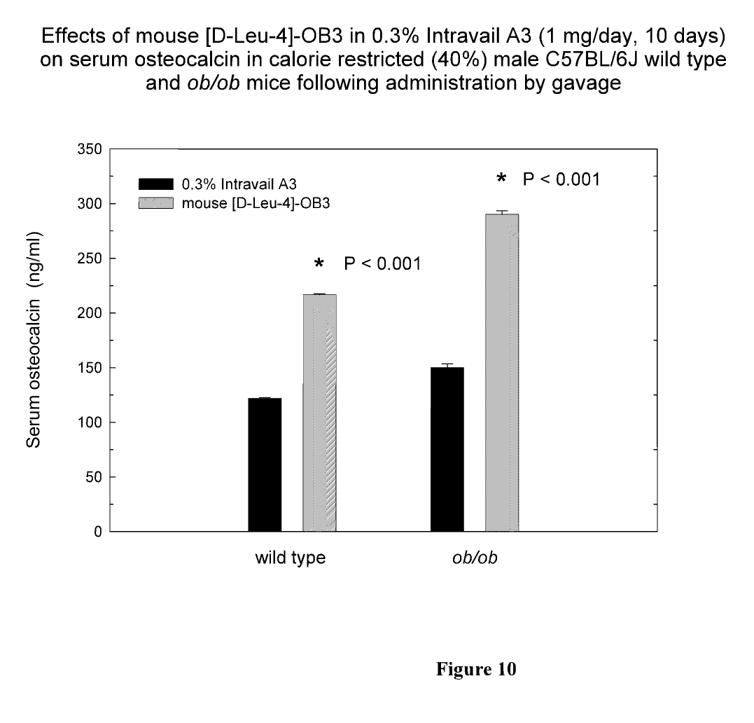

Calorie restriction reduced osteocalcin levels in both wild type and ob/ob mice by 44.2% and 19.1%, respectively, when compared to wild type and ob/ob mice allowed food and water ad libitum. Treatment of calorie restricted wild type mice with mouse [D-Leu-4]-OB3 for 10 days significantly (P<0.001) elevated serum osteocalcin levels to levels Seen in Intravail® treated wild type mice fed ad libitum. In ob/ob mice, mouse [D-Leu-4]-OB3 significantly (P<0.001) elevated serum osteocalcin by 93.4% after 10 days of treatment (FIG. 10).

The effects of mouse [D-Leu-4]-OB3 in calorie restricted C57BL/6J wild type and ob/ob mice are summarized in Table 2.

TABLE 2
Effects of mouse [D-Leu-4]-OB3 (1 mg/day. 10 days, gavage)
in calorie restricted (40%) male C57BL/6J wild type and ob/ob mice.

| | |
|---|---|
| | Mouse |
| Control | [D-Leu-4]-OB3 |

Wild type

| | | |
|---|---|---|
| Initial body weight (g) | 24.1 ± 0.6 | 22.6 ± 0.8 |
| Final body weight (g) | 21.9 ± 0.8 | 20.3 ± 0.7 |
| Body weight (% of initial) | 91.0 ± 0.8 | 90.0 ± 0.8 |
| Initial serum glucose (mg/dl) | 208.8 ± 29.2 | 186.7 ± 36.7 |
| Final serum glucose (mg/dl) | 148.0 ± 17.5 | 134.8 ± 14.8 |
| Serum osteocalcin (ng/ml) | 122.0 ± 0.8 | 216.6 ± 0.4 |

Ob/ob

| | | |
|---|---|---|
| Initial body weight (g) | 31.4 ± 0.4 | 34.2 ± 0.4 |
| Final body weight (g) | 28.8 ± 0.3 | 30.8 ± 0.6 |
| Body weight (% of initial) | 91.6 ± 0.6 | 90.0 ± 0.8 |
| Initial serum glucose (mg/dl) | 486.5 ± 23.4 | 480.2 ± 36.7 |
| Final serum glucose (mg/dl) | 270.2 ± 42.3 | 176.5 ± 32.8 |
| Serum osteocalcin (ng/ml) | 150.0 ± 1.4 | 290.1 ± 3.2 |

### Example 2

Previous work with leptin-related synthetic peptides indicated that the entire leptin molecule is not required for the expression of its biological activity. (See Grasso P. et al., Regulatory Pept. 101:123-9 (2001); Grasso P. et al., Regulatory Pept. 85:93-100 (1999); Grasso P. et al., Endocrinology 138:1413-8 (1997); Grasso P. et al., Diabetes 48:2204-9 (1999); and Rozhayskaya-Arena M. et al., Endocrinology 141:2501-7 (2000)). Similar results have been consistently reported by other laboratories utilizing both in vivo and in vitro approaches, peripheral and intracerebroventricular delivery systems, and different animal models. (See Gonzalez L C. et al., Neuroendocrinology 70:213-20 (1999); Malendowicz L K. et al., A. Med. Sci. Res. 27:675-6 (1999); Tena-Sempere M. et al., Eur. J. Endocrinol. 142:406-10 (2000); Malendowicz L K. et al., Endocr. Res. 26:109-18 (2000); Malendowicz L K. et al., J. Steroid Biochem. Mol. Biol. 87:265-8 (2003); Markowska A. et al., Int. J. Mol. Med. 13:139-41 (2004); Malendowicz L K. et al., Int. J. Mol. Med. 14:873-7 (2004); Oliveira Jr V X. et al., Regulatory Pept. 127:123-32 (2005); Oliveira Jr. V X. et al., J. Pept. Sci. 14:617-25 (2008); and Martins M N C. et al., Regulatory Pept. 153:71-82 (2009)). Thus, it has become clear that synthetic peptide analogs which encompass the functional domain of leptin carry sufficient information to influence leptin-modulated physiologies by pathways that may either augment, complement, or diverge from (see Grasso P. et al., Diabetes 48:2204-9 (1999)) those of endogenous leptin. In light of the inconsistent results of leptin management of human obesity in the clinical setting, these observations in rodents may have significant relevance to the development of leptin-related drug therapies that target the treatment of human obesity and its related disorders.

More recently, it has been shown that Intravail®, a patented transmucosal absorption enhancement agent, significantly improves the uptake and bioavailability of mouse [D-Leu-4]-OB3 (See U.S. Pat. Nos. 6,777,388; 7,186,694; 7,208,572B2; Australian Patent number 772278), a bioactive leptin-related synthetic peptide amide, when delivered intranasally. (See Novakovic Z. et al., Regulatory Pept 154:107-11 (2009)). The biphasic absorption profile observed in this study suggested that in addition to the initial rapid transport of mouse [D-Leu-4]-OB3 across the nasal mucosa, a later gastrointestinal phase of peptide uptake occurs. This study presents evidence demonstrating that, following oral delivery of mouse [D-Leu-4]-OB3, gastrointestinal absorption occurs with a time course similar to that Seen for the later peak in the biphasic uptake profile associated with intranasal delivery. Moreover, delivery of mouse [D-Leu-4]-OB3 in Intravail® greatly enhances this uptake. The biological activity of intranasally delivered mouse [D-Leu-4]-OB3 in Intravail® in db/db mice, and in wild type and ob/ob mice following oral administration has been confirmed. (See Maggio E T. Expert Opin. Drug Deliv. 3:529-39 (2006)).

### Materials And Methods

### Animal Procedures

### Housing

Six week-old male Swiss Webster mice weighing approximately 30 g were obtained from Taconic Farms (Germantown, N.Y., USA). The animals were housed three per cage in polycarbonate cages fitted with stainless steel wire lids and air filters, and supported on ventilated racks (Thoren Caging Systems, Hazelton, Pa., USA) in the Albany Medical College Animal Resources Facility. The mice were maintained at a constant temperature (24° C.) with lights on from 07:00 to 19:00 h, and allowed food and water ad libitum until used for uptake studies.

### Peptide Administration

Mouse [D-Leu-4]OB3 was prepared commercially as a C-terminal amide by Bachem (Torrance, Calif., USA). For oral delivery, mouse [D-Leu-4]-OB3 was dissolved in either phosphate buffered saline (PBS, pH 7.2) or 0.3% Intravail® (Aegis Therapeutics, San Diego, Calif. USA) reconstituted in water, at a concentration of 1 mg/200 ul and administered by gavage. Is has been previously shown that this concentration to be optimum for regulating energy expenditure, glycemic control, and insulin sensitivity in two genetically obese mouse models. (See Grasso P. et al., Regulatory Pept. 101:123-9 (2001); Grasso P. et al., Regulatory Pept. 85:93-100 (1999); Grasso P. et al., Endocrinology 138:1413-8 (1997); Grasso P. et al., Diabetes 48:2204-9 (1999); and Rozhayskaya-Arena M. et al., Endocrinology 141:2501-7 (2000)). At time zero (0), 200 μl mouse [D-Leu-4]-OB3 in PBS or 0.3% Intravail® was delivered by gavage to each mouse. Following peptide administration, the mice were transferred to separate cages for the designated time period.

### Collection of Blood and Serum Preparation

Five, 10, 20, 40, 60, or 120 min after peptide delivery, the mice (six per time point) were anesthetized with isoflurane (5%) and exsanguinated by cardiac puncture. Euthanasia was confirmed by cervical dislocation. The blood was collected in sterile nonheparinized plastic centrifuge tubes and allowed to stand at room temperature for 1 h. The clotted blood was rimmed from the walls of the tubes with sterile wooden applicator sticks. Individual serum samples were prepared by centrifugation for 30 min at 2600×g in an Eppendorf 5702R, A-4-38 rotor (Eppendorf North America, Westbury, N.Y., USA), The serum samples in each experimental group (n=6) were pooled and stored frozen until assayed for mouse [D-Leu-4]-OB3 content by competitive ELISA.

All of these animal procedures were approved by the Albany Medical College Animal Care and Use Committee, and were performed in accordance with relevant guidelines and regulations.

### Mouse [D-Leu-4]-OB3 Competitive ELISA

Mouse [D-Leu-4]-OB3 content of the pooled serum samples was measured by a competitive ELISA developed and validated in our laboratory as previously described. (See Novakovic Z. et al., Regulatory Pept. 154:107-11 (2009)).

### Pharmacokinetic Analyses

### Relative Bioavailability

Serum concentrations of mouse [D-Leu-4]OB3 vs. time following oral delivery were plotted using the graphics program SigmaPlot 8.0 (SPSS Science, Chicago, Ill., USA). The area under the curve (AUC) was calculated with a function of this program. The lowest AUC value obtained was arbitrarily set at 1.0. Relative bioavailabilty was determined by comparing all other AUC values to 1.0.

Serum Half-Life (t_{1/2})

The period of time required for the serum concentration of mouse [D-Leu-4]-OB3 to be reduced to exactly one-half of the maximum concentration achieved following oral administration of mouse [D-Leu-4]-OB3 in the absence or presence of Intravail® was calculated using the following equation:

t_{1/2}=0.693/ₖₑₗᵢₘ

where kₑₗᵢₘ represents the elimination constant, determined by plotting the natural log of each of the concentration points in the beta phase of the uptake profile against time. Linear regression analysis of these plots resulted in a straight line, the slope of which correlates to the kₑₗᵢₘ.

### Plasma Clearance (CL)

Clearance of mouse [D-Leu-4]-OB3 from the plasma following oral delivery was calculated from the AUC using the following equation:

CL=Dose/AUC

### Apparent Volume of Distribution (V_{d})

Since the half-life of a drug is inversely related to its clearance from the plasma and directly proportional to its volume of distribution, the apparent volume of distribution of mouse [D-Leu-4]-OB3 following oral delivery was calculated from its half-life and clearance using the following equation:

t_{1/2}=0.693×V_{d}/CL

### Results

### Uptake Profile

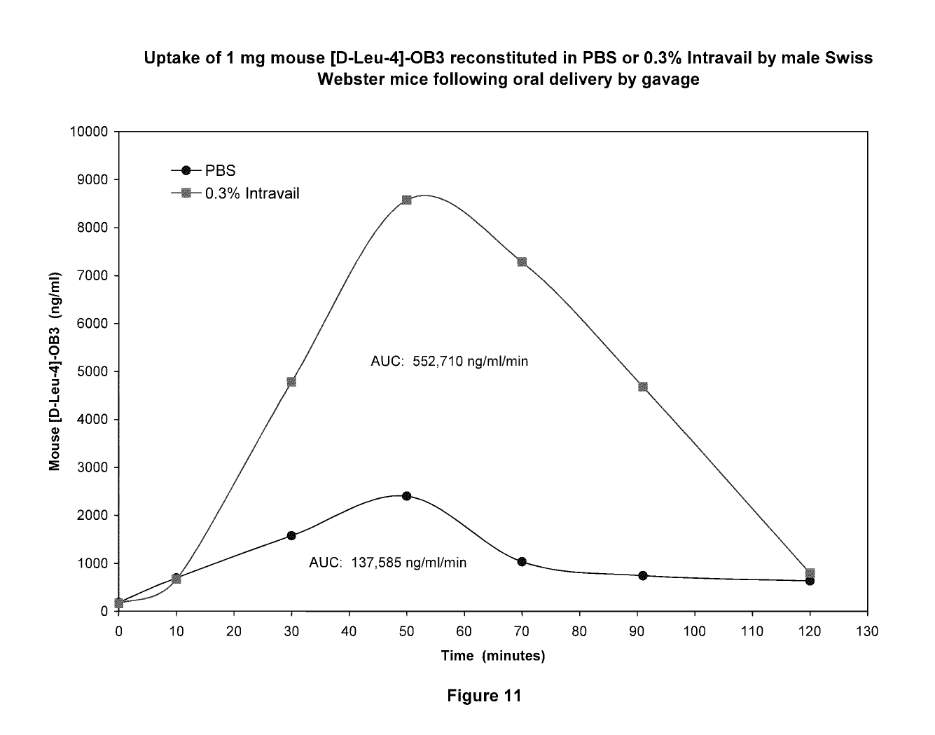

The uptake profiles of mouse [D-Leu-4]-OB3 following oral delivery in the absence or presence of Intravail® are shown in FIG. 11. Maximum uptake (Cₘₐₓ) of 1 mg of mouse [D-Leu-4]-OB3 reconstituted in 0.3% Intravail® was 3.6-fold greater than that Seen when the peptide was delivered in PBS (8574 ng/ml vs. 2400 ng/ml, respectively). Maximum uptake (Tₘₐₓ) occurred at 50 min in both cases. Serum concentrations of mouse [D-Leu-4]-OB3 decreased with time at different rates.

### Relative Bioavailability

The relative bioavailability of orally delivered mouse [D-Leu-4]-OB3 in the absence or presence of Intravail® was determined by measuring the area under the uptake curve (AUC). This value represents the total extent of peptide absorption into the systemic circulation. The AUC values following oral delivery of 1 mg mouse [D-Leu-4]-OB3 in the absence or presence of Intravail® were 137,585 ng/ml/min and 552,710 ng/ml/min, respectively. From these values, the relative bioavailabilities were calculated to be 1.0 and 4.0, respectively.

Serum Half-Life (t_{1/2})

The serum half-life of mouse [D-Leu-4]-OB3 following oral delivery in PBS or Intravail® was inversely correlated with the elimination constants calculated as described above (Table 3). The serum half-life of mouse [D-Leu-4]-OB3 delivered in PBS was determined to be 36.86 min with a kₑₗᵢₘ of 0.0188 ml/min while that of mouse [D-Leu-4]-OB3 delivered in Intravail® was 20.15 min with a kₑₗᵢₘ of 0.0344 ml/min. Plasma clearance (CL) and apparent volume of distribution (Vd)

Plasma CL of mouse [D-Leu-4]-OB3 delivered in PBS was four times faster than that calculated for Intravail® (7.22 ml/min and 1.81 ml/min, respectively). The apparent volume of distribution of mouse [D-Leu-4-]OB3 following delivery in PBS or Intravail® was calculated using the half-life and clearance rates previously calculated, and was determined to be 71.45 ml and 49.74 ml, respectively. The V_{d} of mouse [D-Leu-4]-OB3 in the absence or presence of Intravail® was directly correlated with serum half-life (Table 3).

All pharmacokinetic parameters measured in this study are summarized in Table 3.

TABLE 3
Pharmacokinetic parameters of mouse [D-Leu-4]-OB3 uptake
in male Swiss Webster mice following oral delivery (by gavage)
of 1 mg of peptide reconstituted in PBS or Intravail ®.

| Parameter | PBS | Intravail ® |
|---|---|---|
| Cₘₐₓ (ng/ml) | 2400 | 8574 |
| tₘₐₓ (min) | 50 | 50 |
| AUC (ng/ml/min) | 137,585 | 552,710 |
| Relative bioavailability | 1.0 | 4.0 |
| kₑₗᵢₘ (ml/min) | 0.0188 | 0.0344 |
| t_{1/2} (min) | 6.86 | 20.15 |
| CL (ml/min) | 7.22 | 1.81 |
| V_{d} (ml) | 71.45 | 49.74 |

### Relative Oral Bioavailability of Mouse [D-Leu-4]-OB3 in Intravail® Compared to Intranasal Administration and Commonly Used Injection Modes of Delivery

The relative oral bioavailability of mouse [D-Leu-4]-OB3 delivered in Intravail® was compared to the relative bioavailabilities of intranasal and three commonly used injection methods of delivery recently reported. (See Oliveira Jr. V X. et al., J. Pept. Sci. 14:617-25 (2008)). This was done by comparing the AUC of orally delivered mouse [D-Leu-4]-OB3 in Intravail® to the AUC of mouse [D-Leu-4]-OB3 reconstituted in PBS and delivered by ip, sc, and im injection, and to the AUC of mouse [D-Leu-4]-OB3 (in Intravail®) following intranasal delivery. The relative oral bioavailability of mouse [D-Leu-4]-OB3 compared to each of the other modes of delivery is expressed as a percent. This data is presented in Table 4.

TABLE 4
Relative oral bioavailability of mouse [D-Leu-
4]-OB3 in Intravail ® compared to injection
and intranasal modes of administration.

| | | Relative oral |
|---|---|---|
| Method of delivery | AUC (ng/ml/min) | bioavailability (%) |
| Oral (by gavage) | 559,330 | |
| Intraperitoneal | 1,072,270^{a} | 52.2 |
| Subcutaneous | 1,182,498^{a} | 47.3 |
| Intramuscular | 1,481,060^{a} | 37.8 |
| Intranasal | 4,336,963^{a} | 12.9 |

^{a}value taken from reference [Novakovic Z. et al, Regulatory Pept. 54: 107-11 (2009)]

### Example 3

### Intranasal Delivery of Mouse [D-Leu-4]-OB3, a Synthetic Peptide Amide with Leptin-Like Activity, in Male C57BLK/6-m db/db Mice: Effects on Energy Balance, Serum Osteocalcin, and Serum Insulin Levels

It has recently shown that intranasal administration of mouse [D-Leu-4]-OB3 reconstituted in Intravail® to male Swiss Webster mice resulted in significantly higher bioavailability than commonly used injection methods of delivery. The absorption profile associated with intranasal delivery of mouse [D-Leu-4]-OB3 showed an early peak representing absorption across the nasal mucosa, and a later peak suggesting a gastrointestinal site of uptake.

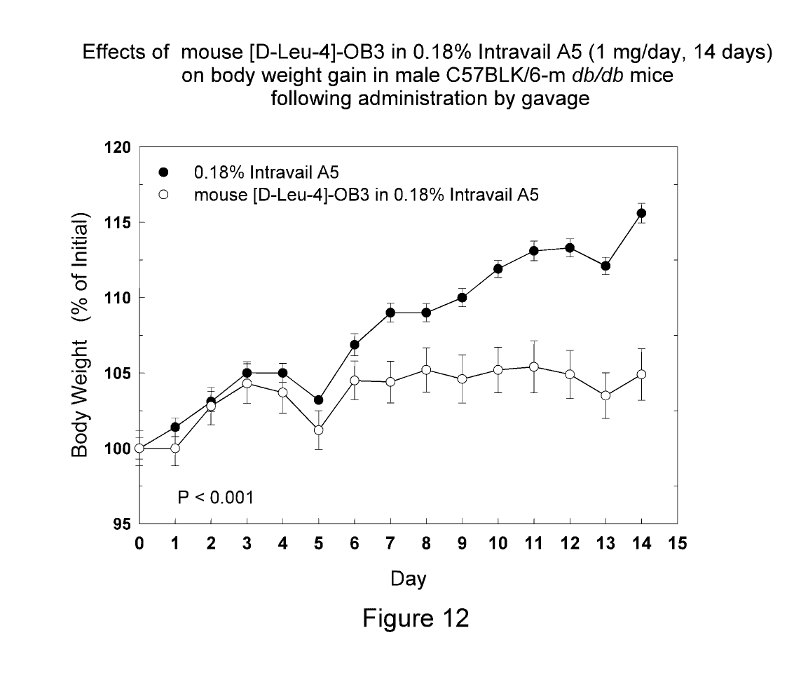

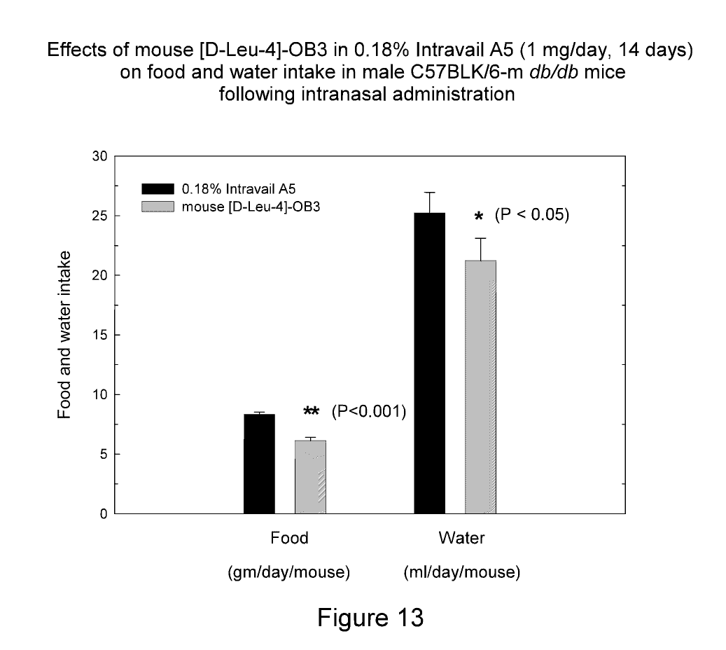

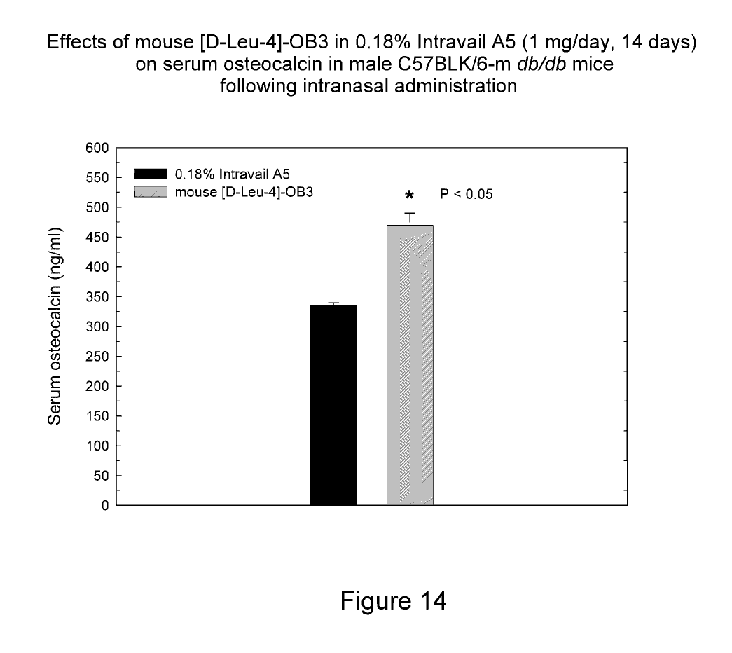

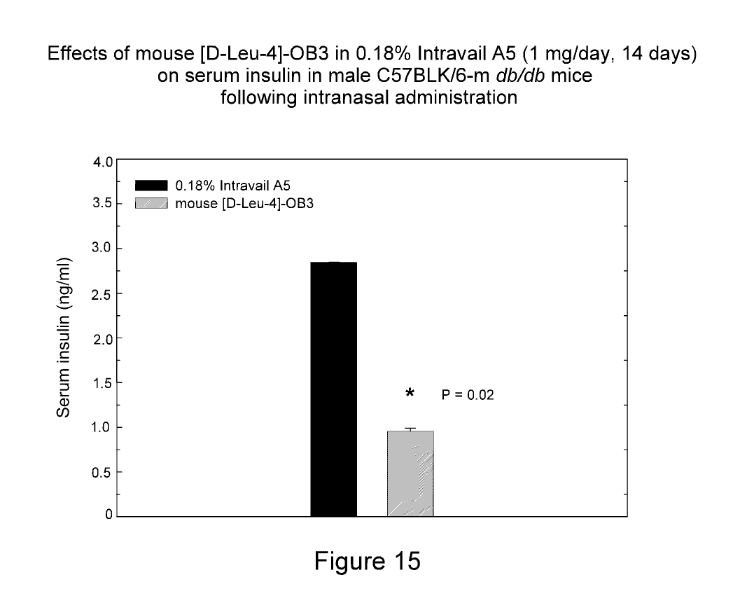

In the present study, the effects of intranasal administration of mouse [D-Leu-4]-OB3 in Intravail® on energy balance, serum osteocalcin, and serum insulin levels in genetically obese male C57BLK/6-m db/db mice allowed food and water ad libitum were examined. Treatment with mouse [D-Leu-4]-OB3 reduced body weight gain, food intake, and water intake by 10.7%, 16.5%, and 11.9%, respectively. (See FIGS. 12 and 13). Serum osteocalcin levels in db/db mice treated with mouse [D-Leu-4]-OB3 were elevated by 161.0% over controls, and serum insulin levels in db/db mice treated with mouse [D-Leu-4]-OB3 were approximately 3-fold lower than those in untreated controls. (See FIGS. 14 and 15). These data indicate that intranasal delivery of biologically active mouse [D-Leu-4]-OB3 in Intravail® is possible and that it has significant effects on regulating body weight gain, food and water intake, bone formation, and hyperinsulinemia, Taken together, these results suggest that intranasal delivery of mouse [D-Leu-4]-OB3 may have potential not only as an alternative therapy in the treatment of human obesity and some of its associated metabolic dysfunctions but also as a means to prevent and/or reverse at least some of the bone loss which accompanies osteoporosis, anorexia nervosa, and other wasting diseases.

### Example 4

### Intranasal Delivery of Mouse [D-Leu-4]-OB3, a Synthetic Peptide Amide with Leptin-Like Activity, Improves Energy Balance, Glycemic Control, Insulin Sensitivity, and Bone Formation in Leptin-Resistant C57BLK/6-m db/db Mice

It has recently been shown that intranasal administration of mouse [D-Leu-4]-OB3 reconstituted in Intravail® to male Swiss Webster mice resulted in significantly higher uptake and bioavailability when compared to commonly used injection methods of delivery. In the present study, the effects of intranasal delivery of mouse [D-Leu-4]-OB3 in Intravail® on energy balance, glucose regulation, insulin secretion, and serum levels of osteocalcin, a specific and sensitive marker of bone formation were examined Genetically obese C57BLK/6-m db/db mice were allowed food and water ad libitum, and given either Intravail® alone or mouse [D-Leu-4]-OB3 in Intravail® for 14 days. Mouse [D-Leu-4]-OB3 reduced body weight gain, daily food intake, daily water intake, and serum glucose by 11.5%, 2.2%, 4.0%, and 61.9%, respectively. Serum insulin levels in db/db mice given mouse [D-Leu-4]-OB3 were approximately 3-fold lower than those in mice receiving Intravail® alone. Mouse [D-Leu-4]-OB3 elevated serum osteocalcin in db/db mice by 28.7% over Intravail® treated control mice. These results indicate that intranasal delivery of biologically active mouse [D-Leu-4]-OB3 in Intravail® is feasible, and has significant effects on regulating body weight gain, food and water intake, serum glucose, insulin sensitivity, and bone formation. Moreover, the observed effects of mouse [D-Leu-4]-OB3 on energy balance, glycemic regulation, and insulin sensitivity further suggest that intranasal delivery of mouse [D-Leu-4-OB3 may have potential therapeutic application to the treatment of both human obesity and type 2 diabetes mellitus, in the absence or presence of an obese background. In addition, its effects on bone turnover may also be useful in the prevention and/or reversal of at least some of the bone loss which accompanies osteoporosis, anorexia nervosa, cancer, and other wasting diseases not associated with the obesity syndrome.

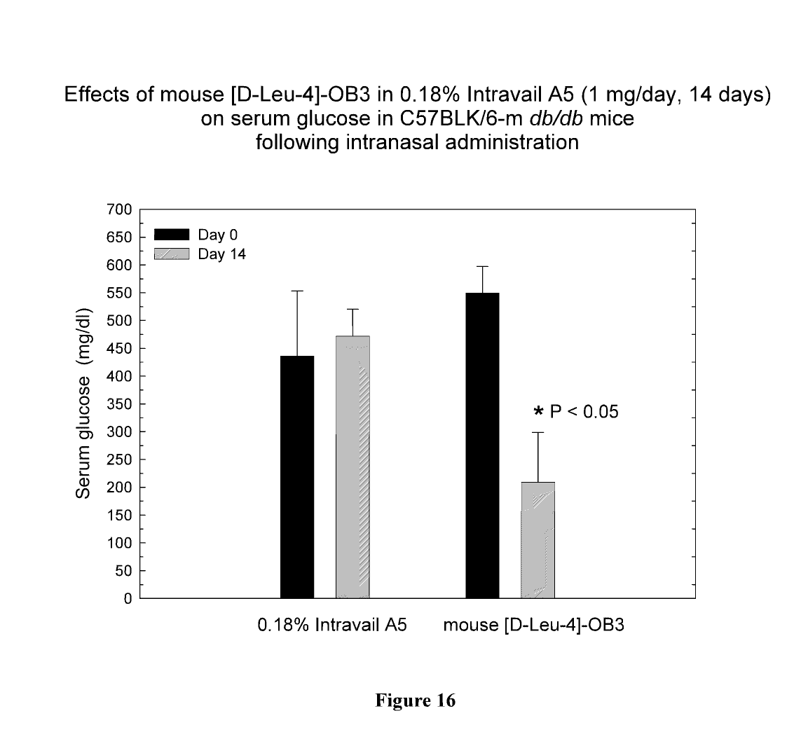

The effects of mouse [D-Leu-4]-OB3 on serum glucose levels in db/db mice are shown in FIG. 16. In mice receiving Intravail® alone for 14 days, serum glucose levels were essentially the same. After 14 days of treatment with mouse [D-Leu-4]-OB3, serum glucose levels were significantly (P<0.05) reduced by 61.9%.

The anorexogenic activity, effects on body weight gain, glycemic regulation, insulin sensitivity, bone turnover, and lack of toxicity of mouse [D-Leu-4]-OB3 in both leptin-deficient ob/ob and leptin-resistant db/db mice makes this peptide an attractive candidate for drug development for the treatment not only of human obesity, but also for type 2 diabetes mellitus, osteoporosis, and other wasting diseases.

In the present study, it has been shown that the biological activity of mouse [D-Leu-4]-OB3 is retained in leptin-resistant C57BLK/6-m db/db mice when it is delivered by intranasal instillation in Intravail®Similar results were seen after oral administration of mouse [D-Leu-4]-OB3 in Intravail® to C57BL/6J leptin-deficient ob/ob mice. (See Lee et al., Regulatory Pept 160:129-32 (2010)). The pharmacokinetics of both intranasal (see Novakovic et al., Regulatory Pept 154:107-11 (2009)) and oral (see Lee et al., Regulatory Pept 160:129-32 (2010)) uptake of mouse [D-Leu-4]-OB3 in Intravail® have been previously described.

Worthy of special note are the robust effects of intranasal delivery of mouse [D-Leu-4]-OB3 in Intravail® on glycemic control and insulin sensitivity that are reported here. In an earlier pair-feeding study with ob/ob mice, it was shown that restriction of caloric intake alone could not account for the pronounced anti-hyperglycemic and anti-hyperinsulinemic effects of mouse [D-Leu-4]-OB3 when delivered by i.p. injection. These results indicate that mouse [D-Leu-4]-OB3 exerts its influence on serum glucose levels not only by suppressing caloric intake, but also through a separate and distinct action on glucose metabolism. In the present study, similar effects on serum glucose and insulin levels were seen in db/db mice when mouse [D-Leu-4]-OB3 was delivered by nasal instillation.

The results summarized below we show that intranasal delivery of mouse [D-Leu-4]-OB3 to genetically obese C57BLK/6-m db/db mice has similar effects on energy balance, glycaemic control, insulin sensitivity and bone formation as seen in ob/ob mice following i.p. or oral administration. Given the fact that the majority of clinically obese humans are leptin resistant [Lonnqvist et al. Nat Med 1997; 1: 950-953] because of defects in transport of leptin across the blood-brain barrier (BBB) [Banks W A. Curr Phar Des 2008; 14: 1606-1614], the relevance of these results in a leptin-resistant rodent model of obesity to the clinical management of human obesity may be highly significant.

### Results

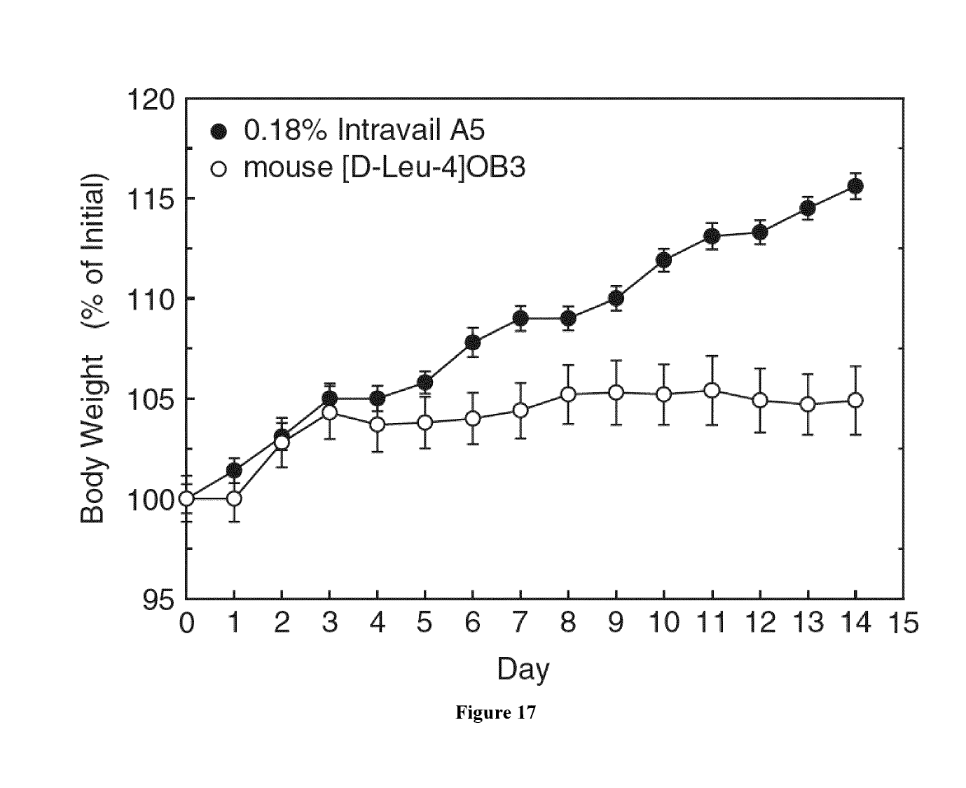

Effects on Body Weight Gain. The effects of intranasal delivery of mouse [D-Leu-4]-OB3 on body weight gain in db/db mice allowed food and water ad libitum are shown in FIG. 17. After 14 days of receiving Intravail® alone, db/db mice were 16.4% heavier than they were at the beginning of the study. Mice treated with mouse [D-Leu-4]-OB3 were only 4.9% heavier than their initial body weight and were significantly lighter (p<0.001) than their untreated counterparts.

Effects on Food and Water Intake. Daily food intake of db/db mice treated with mouse [D-Leu-4]-OB3 was significantly (p<0.001) less when compared with db/db mice receiving Intravail® alone (6.1 vs. 8.3 g/mouse/day, respectively). Daily water consumption by db/db mice receiving mouse [D-Leu-4]-OB3 was also significantly (p<0.05) less compared with Intravail® treated controls (21.2 ml/mouse/day vs. 25.2 ml/mouse/day, respectively).

Effects on Serum Glucose Levels. In mice receiving Intravail® alone for 14 days, final serum glucose levels were approximately the same as they were at the beginning of the study. After 14 days of treatment with mouse [D-Leu-4]-OB3, serum glucose levels were significantly (p<0.001) reduced by 61.9%.

Effects on Serum Insulin Levels. Intranasal administration of mouse [D-Leu-4]-OB3 to db/db mice for 14 days significantly (p<0.01) reduced serum insulin levels. The serum insulin concentration of mice receiving mouse [D-Leu-4]-OB3 was approximately threefold lower than in Intravail® treated control mice (0.95 vs. 2.85 ng/ml, respectively).

Effects on Serum Osteocalcin Levels. Treatment of db/db mice with mouse [D-Leu-4]-OB3 for 14 days resulted in significantly (p<0.05) elevated serum osteocalcin levels when compared with Intravail® treated control mice. Serum osteocalcin levels in mice receiving mouse [D-Leu-4]-OB3 were 28.7% higher than their Intravail® treated counterparts (470 vs. 335 ng/ml, respectively). The effects of mouse [D-Leu-4]-OB3 in C57BLK/6-m db/db mice are summarized in Table 4-1.

TABLE 4-1
Effects of mouse [D-Leu-4]-OB3 (1 mg/day; 14 days, intranasal
delivery) in leptin-resistant C57BLK/6-m db/db mice.

| | | Mouse |
|---|---|---|
| | Intravail ® | [D-Leu-4]-OB3 |
| Initial body weight (g) | 35.2 ± 0.6 | 35.0 ± 1.1 |
| Final body weight (g) | 41.0 ± 0.6 | 37.0 ± 1.7 |
| Body weight (% of initial) | 116.4 ± 0.6 | 104.9 ± 1.4 |
| Food intake (g/mouse/day) | 8.3 ± 0.2 | 6.1 ± 0.3 |
| Water intake (ml/mouse/day) | 25.2 ± 1.7 | 21.2 ± 1.8 |
| Initial serum glucose (mg/dl) | 436 ± 117. | 472 ± 7.3 |
| Final serum glucose (mg/dl) | 549 ± 47.8 | 209 ± 89.4 |
| Serum insulin (ng/ml) | 2.85 ± 0.02 | 0.95 ± 0.03 |
| Serum osteocalcin (ng/ml) | 335.0 ± 5.0 | 470.0 ± 20.0 |

These results show that the biological activity of mouse [D-Leu-4]-OB3 is retained in leptin-resistant C57BLK/6-m db/db mice when it is delivered by intranasal instillation in Intravail®. Although the mechanism of action by which mouse [D-Leu-4]-OB3 influences leptin-modulated physiologies in both ob/ob and db/db mice is unclear at present, the reproducibility of our earlier results with bioactive leptin-related peptides in db/db mice following i.p. injection and in ob/ob mice following i.p. and oral delivery is undeniable. Given the redundancy and interplay of hypothalamic regulators of energy balance, the possibility of peptide activation of another as yet unidentified signal transducing isoform of the leptin receptor cannot be discounted. Alternatively, mouse [D-Leu-4]-OB3 may augment the effects of some other energy regulatory ligand with its receptor, such as α-MSH interaction with the melanocortin-4 receptor (MC4-R). This latter mechanism is currently under investigation in our laboratory.

The effects of mouse [D-Leu-4]-OB3 on serum osteocalcin levels shown in the present study, together with similar results in studies with orally delivered mouse [D-Leu-4]-OB3 in an ob/ob mouse model (see Novakovic et al., Diabetes, Obesity and Metabolism 2009 (in press), suggest that the mechanism by which mouse [D-Leu-4]-OB3 exerts its effects on glycemic regulation and insulin sensitivity in both ob/ob and db/db mice may be related to its ability to elevate serum osteocalcin levels. Osteocalcin, a hormone produced by mature osteoblasts, acts not only as a sensitive and specific marker of osteoblastic activity and bone formation, but also enhances glucose utilization in peripheral tissues as a result of increased insulin sensitivity. The data presented here provide evidence to support this mechanism of action.

The ability of mouse [D-Leu-4]-OB3 to elevate serum osteocalcin levels shown in this study, together with similar results in our earlier study with orally delivered mouse [D-Leu-4]-OB3 in an ob/ob mouse model [Novakovic et al. Diabetes Obes Metab 2010; 12: 532-539, incorporated by reference herein in its entirety], suggests that the mechanism by which mouse [D-Leu-4]-OB3 exerts its effects on glycaemic regulation and insulin sensitivity in both ob/ob and db/db mice may be related, at least in part, to its ability to elevate serum osteocalcin levels. Osteocalcin, a hormone produced by mature osteoblasts, acts not only as a sensitive and specific marker of osteoblastic activity and bone formation but also enhances glucose utilization in peripheral tissues as a result of increased insulin sensitivity.

If elevation of serum osteocalcin is the mechanism by which [D-Leu-4]-OB3 exerts its effects on glycaemic control, then the potential usefulness of intranasal or oral delivery of [D-Leu-4]-OB3 in Intravail® as a novel therapeutic approach to the treatment of type 2 diabetes in humans, in the absence or presence of an obese background, may be of great clinical significance.

Enhancement of serum osteocalcin was observed in this study following intranasal delivery of mouse [D-Leu-4]-OB3 to leptin-resistant db/db mice. These data indicate that the anabolic effects of i.p. leptin on bone turnover can be achieved by mouse [D-Leu-4]-OB3 when delivered by nasal instillation or orally in Intravail®.

While there may be the possibility of an influence of isoflurane on metabolic control in these mice that might limit interpretation of our data, the significant differences between Intravail® treated control mice and mice receiving mouse [D-Leu-4]-OB3, in body weight gain, food and water intake, blood glucose and serum insulin and osteocalcin levels, suggest that these effects are peptide related. Moreover, similar changes in these parameters were also observed after oral delivery of mouse [D-Leu-4]-OB3, in the absence of isoflurane, to C57BL/6J ob/ob mice. Novakovic et al. Diabetes Obes Metab 2010; 12: 532-539, incorporated by reference herein in its entirety.

The usefulness of [D-Leu-4]-OB3 reformulated in Intravail®, either as a monotherapy or in combination with other regulators of energy balance, may offer a promising approach to the management of human obesity and its associated metabolic disorders in the clinic. Furthermore, the observed effects of mouse [D-Leu-4]-OB3 on glycaemic control, insulin sensitivity and bone turnover in mice following oral or intranasal delivery in Intravail® suggest that this peptide may be used as a therapeutic alternative for the treatment of other chronic diseases in humans, such as type 2 diabetes mellitus, osteoporosis and bone loss associated with other wasting diseases.

### EQUIVALENTS

From the foregoing detailed description of the specific embodiments of the invention, it should be apparent that unique compositions and methods of use for synthetic leptin-related peptides have been described. Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of compositions and methods of use of synthetic leptin-related peptides, including type of amino acid derivative to be incorporated, is believed to be a matter of routine for a person of ordinary skill in the art with knowledge of the embodiments described herein. Other aspects, advantages, and modifications considered to be within the scope of the following claims. The claims presented are representative of the inventions disclosed herein. Other unclaimed inventions within this disclosure are also contemplated. Applicants reserve the right to pursue such inventions in later claims.

What is claimed is:

1. A method of increasing bone formation comprising administering to a subject in need thereof a therapeutically effective amount of a pharmaceutical composition comprising a leptin peptide and a pharmaceutically acceptable carrier, wherein the leptin peptide is between 7 and 15 amino acids in length and comprises the amino acid sequence of SEQ ID NO:2 or SEQ ID NO:18.

2. The method of claim 1, wherein the leptin peptide is SEQ ID NO:2.

3. The method of claim 1, wherein the leptin peptide is SEQ ID NO:18.

4. The method of claim 1, wherein the step of administering to a subject comprises oral, anal, injection, or intranasal administration.

5. The method of claim 1, wherein increases in bone formation is measures by increases in serum osteocalcin levels.

6. The method of claim 1, wherein the subject is a human.

7. The method of claim 1, wherein the pharmaceutically acceptable carrier is an alkylglycoside, wherein the alkylglycoside is selected from the group consisting of dodecyl maltoside, tridecyl maltoside, sucrose mono-dodecanoate, sucrose mono-tridecanoate, and sucrose mono-tetradecanoate.

8. The method of claim 1, wherein the pharmaceutically acceptable carrier is a nontoxic, nonionic alkyl glycoside having a hydrophobic alkyl joined by a linkage to a hydrophilic saccharide, wherein the alkyl has from 9 to 24 carbons.

9. The method of claim 8, wherein the saccharide is selected from the group consisting of maltose, sucrose and glucose, and wherein the linkage is selected from the group consisting of a glycosidic linkage, a thioglycosidic linkage, an amide linkage, a ureide linkage and an ester linkage.

10. The method of claim 1, wherein the leptin peptide is a purified peptide which is an OB3 peptide consisting of amino acid residues ¹¹⁶ Ser-Cys-Ser-Leu-Pro-Gln-Thr¹²² of mouse leptin protein (SEQ ID NO:2) or ¹¹⁶Ser-Cys-His-Leu-Pro-Trp-Ala¹²² of human leptin protein (SEQ ID NO:18).

11. The method of claim 1, wherein one to seven amino acids of the leptin peptide is substituted with its corresponding D-amino acid isoform.

12. The method of claim 1, wherein the subject suffers from a disorder selected from the group consisting of malnutrition, starvation, anorexia nervosa, osteoporosis, cancer, diabetes, tuberculosis, chronic diarrhea, AIDS, and Superior mesenteric artery syndrome.

13. A method of treating a wasting disease comprising administering to a subject suffering therefrom a therapeutically effective amount of a pharmaceutical composition comprising a leptin peptide of SEQ ID NO:2 or SEQ ID NO:18 and a pharmaceutically acceptable carrier, wherein the leptin peptide increases serum osteocalcin levels in said subject.

14. The method of claim 13, wherein the wasting disease is selected from the group consisting of malnutrition, starvation, anorexia nervosa, osteoporosis, cancer, diabetes, tuberculosis, chronic diarrhea, AIDS, and Superior mesenteric artery syndrome.

15. The method of claim 13, wherein the step of administering to a subject comprises oral or intranasal administration.

16. The method of claim 13, wherein the pharmaceutical composition is in the form of a capsule, a tablet, a quick dissolving film, a liquid, nosedrops, a spray, or a suppository.

17. The method of claim 13, wherein the leptin peptide is a purified peptide which is an OB3 peptide consisting of amino acid residues ¹¹⁶ Ser-Cys-Ser-Leu-Pro-Gln-Thr¹²² of mouse leptin protein (SEQ ID NO:2) or ¹¹⁶Ser-Cys-His-Leu-Pro-Trp-Ala¹²² of human leptin protein (SEQ ID NO:18).

18. The method of claim 13, wherein one to seven amino acids of the leptin peptide is substituted with its corresponding D-amino acid isoform.

19. The method of claim 13, wherein the pharmaceutically acceptable carrier is an alkylglycoside, wherein the alkylglycoside is selected from the group consisting of dodecyl maltoside, tridecyl maltoside, sucrose mono-dodecanoate, sucrose mono-tridecanoate, and sucrose mono-tetradecanoate.

20. The method of claim 13, wherein the pharmaceutically acceptable carrier is a nontoxic, nonionic alkyl glycoside having a hydrophobic alkyl joined by a linkage to a hydrophilic saccharide, wherein the alkyl has from 9 to 24 carbons.

21. The method of claim 20, wherein the saccharide is selected from the group consisting of maltose, sucrose and glucose.

22. The method of claim 21, wherein the linkage is selected from the group consisting of a glycosidic linkage, a thioglycosidic linkage, an amide linkage, a ureide linkage and an ester linkage
